(19) United States
(12) Patent Application Publication
(10) Pub. No.: US 2003/0096307 A1
(43) Pub. Date: May 22, 2003

(54) Functional site profiles for proteins and methods of making and using the same

(76) Inventor: Jacquelyn Fetrow, San Diego, CA (US)

Correspondence Address: Bradford J. Duft, Brobeck, Phleger & Harrison LLP, 12390 El Camino Real, San Diego, CA, 92130, US

(21) Appl. No.: 10/199,901

(22) Filed: Jul. 20, 2002

Related U.S. Application Data
(60) Provisional application No. 60/307,425, filed on Jul. 21, 2001.

(51) Int. Cl. G01N 33/53; G06F 19/00; G01N 33/48; G01N 33/50

(52) U.S. Cl. 435/7.1; 702/19

(57) ABSTRACT

Described are functional site profiles, and methods of making and using the same. Specifically, the methods employ functional site profiling to examine the chemistry and structure of functional sites in proteins. Such methods allow classification of proteins, and protein relatedness, based on functional site chemistry and structure, rather than sequence similarity or structural homology across complete protein sequences and structures. Functional site profiling of amino acid residues located in the spatial environment of key functional features allows conserved similarities and characteristic differences in functional sites across protein families to be assessed, which has application not only in terms of protein classification, but also in the context of drug discovery, protein engineering, etc.

### RELATED APPLICATION

This application claims priority to U.S. patent application serial No. 60/307,425, filed Jul. 21, 2001.

### FIELD OF THE INVENTION

This invention generally concerns methods and tools to assess protein biochemical function. More specifically, this invention concerns methods and tools for profiling the chemistry and structure of functional sites in proteins.

### BACKGROUND OF THE INVENTION

1. Introduction

The following description includes information that may be useful in understanding the present invention. It is not an admission that any of the information provided herein is prior art, or relevant, to the presently claimed inventions, or that any publication specifically or implicitly referenced is prior art.

2. Background

The genomics revolution has produced a deluge of gene and protein sequence information. For example, the recent publication of the complete sequence of the human genome revealed approximately 36,000 genes (Venter, et al. (2001), Science, vol. 291: 1304-1351). The sequencing of the genomes of several other multicellular eukaryotic organisms, including the plant Arabidopsis thaliana (approximately 25,500 genes; The Arabidopsis Genome Initiative (2000), Nature, vol. 406: 798-815), and the model organisms Caenorhabditis elegans (about 19,000 genes; The C. elegans Sequencing Consortium (1998), Science, vol. 282: 2012-2018), Drosophila melanogaster (approximately 13,500 genes; Adams, et al. (2000), Science, vol. 287: 2185-2195), and Saccharomyces cerevisiae (about 6,000 genes; http://genome-www.stanford.edu/Saccharomyces), has also recently been reported.

A central tenet in modem biology is that genes encode proteins. In the past 20 years, it has also become clear that the messages that carry the information encoded by the genes (these messages are known as “messenger RNA” or “mRNA” molecules) of multicellular organisms (e.g., eukaryotic organisms such as humans and other mammals, birds, fish, reptiles, insects, and plants) to the cellular machinery that assembles proteins can be differentially processed (e.g., by alternative “splicing” mechanisms), thereby resulting in the production ultimately of possibly several, and perhaps many, proteins from the same gene. As a result, it is expected that the total number of distinct protein types expressed in various tissues of an organism over its life span far exceeds the number of genes, perhaps by a factor of 10 or more. Thus, the proteome, or complete protein complement, of a complex organism is likely to range up to hundreds of thousands or more different proteins, excluding allelic variants and the like. While evolutionary relatedness even between distantly related, taxonomically distinct organisms may provide some level of commonality between the proteomes of different organisms, the total universe of distinct proteins will likely exceed that number by at least several fold.

This exploding quantity of gene and protein data presents a number of challenges. These challenges are perhaps best punctuated by the fact that, in isolation, such information is insufficient to answer the many questions that must be addressed to understand what role(s), if any, a gene and its encoded protein(s) play in a cell, organism, or disease process. The emerging field of proteomics seeks to answer these questions.

One way that proteomics can shed light on the roles that proteins play is to determine biochemical function(s). Researchers have applied various methods in attempting to make these functional determinations. The most common and rapid computational methods use conventional algorithms to perform sequence alignments of complete protein sequences, although these methods are limited by the extent of sequence similarity between sequences of unknown and known function. In such sequence alignment methods, the extent of amino acid sequence identity between an experimental (or “query” or “probe“) sequence and one or more sequences whose function(s) is(are) known is computed. Alignment methods such as BLAST, BLITZ, and FASTA are typically employed for this purpose. Assignment of function is based on the theory that significant sequence identity allows one to infer functional similarity.

However, in part because of the frequent lack of substantial sequence similarity among proteins, these methods often fail. Newly discovered amino acid or nucleotide sequences frequently do not match any known or available sequence. Indeed, many protein amino acid sequences (from 30-60% or more) that have been deduced from genome project-derived nucleotide sequence information represent novel protein families with unknown function, and for which no homologous sequence of known function can be identified. Furthermore, such conventional sequence alignment methods cannot consistently detect functional and structural similarities, particularly when sequence identity is less than about 25-30%. In practice, roughly half of a given genome falls into one of these two categories of no sequence similarity, or less than about 25-30% sequence identity, with a known sequence.

The ability to infer function from sequence similarity is also questionable. Significant inaccuracies, based on function annotation transfer, also have been reported, even at higher levels of sequence identity. The emerging viewpoint is that for sequences with less than about 50% sequence identity, sequence similarity-based annotation transfer is suspect. It is also known that even single amino acid changes can result in total abrogation of protein function. For these reasons, it is clear that alternatives to one-dimensional sequence alignment methods are necessary in order to accurately assess the biochemical function and classification of the vast numbers of amino acid sequences that are being discovered across biology.

In an attempt to overcome some of the problems associated with employing sequence alignments to help predict protein function, several databases of short, local sequence patterns (or “motifs”) have been designed to help identify a given function or activity of a protein. These databases, notably “PROSITE”, “Blocks”, “PRINTS”, and the Hidden Markov Model-derived domain database Pfam, use local sequence information (i.e., the sequence of several contiguous amino acid residues), as opposed to entire amino acid sequences, to try to identify sequence patterns that are specific for a given function. Even then, though, such motif-based annotation methods usually rely upon the ability to identify certain residue patterns observed in multiple sequence alignments of many family members.

Moreover, there is not necessarily a direct correlation between a motif and a given molecular function, or between residue conservation and functional importance (e.g., the residue may be structurally, both functionally, important). Frequently, motifs are applied before the meaning of the motif is known in the context of protein structure. Typically these methods identify multiple sequence motifs for a given protein family that might describe a functional site or domain fold, each applied discretely to a query sequence. A resulting score is calculated on the basis of the number and quality of motif matches found, although interpretation of such scores is often ambiguous and complicated by the realizations that most eukaryotic proteins will carry out multiple functions and that many protein folds are not yet represented in the Protein Data Bank.

Function assignment based on local sequence signatures is also plagued by the deficiencies that limit the use of sequence alignment algorithms to predict protein function. Specifically, as sequence diversity within protein families increases, local sequence signatures may no longer recognize experimental protein sequences as belonging to a functional family. In proteins that are distantly related in terms of evolution, it is expected that only those residues required for the specific biological function (including those required to maintain the necessary three-dimensional structure) of a protein will be conserved. That conservation will include not only sequence conservation, but also three-dimensional structural conservation. However, local sequence motifs cannot recognize conserved three-dimensional structure. Consequently, local sequence motifs often fail to accurately assess protein function because function derives from three-dimensional structure. In other words, local sequence motif analysis is limited when function is dependent upon non-local residues, i.e., amino acids disposed in different regions of a protein's primary structure.

This deficiency is significant, as many functional sites in proteins, particularly in diverse protein families, are known to comprise non-local residues, and these residues are brought into functional association as a result of the protein assuming its folded three-dimensional structure, where different regions of the protein (in terms of linear amino acid sequence) come together. For example, the serine hydrolase family is defined by a common active site triad that carries out hydrolysis, but the protein family has divergent structures, catalytic mechanisms, and substrate specificities. Only a few sparsely distributed sequence positions are well conserved in this (and other) large functional superfamily. Attempts to build a successful consensus pattern for such protein families can fail for various reasons, including limited representation of the functional site and/or inaccuracies in multiple sequence alignments. It has also been recognized that protein families sorted by functional residues alone may not necessarily correlate with global fold family classifications, reiterating the evolutionary disconnect between function sites and overall protein fold.

Alternatives to sequence-based methods include those based on protein structure. As alluded to above, it has been recognized for some time that biochemical function is dictated by the three-dimensional arrangement of specific amino acid residues. Thus, two proteins having the same three-dimensional structure, perhaps not globally but in one or more discrete sub-structures or domains, may be expected to have similar biochemical fictions. As will be appreciated, as between two proteins, small differences in the three-dimensional arrangement of the amino acid residues making up or influencing the functional site, as well as differences amongst the particular amino acid residues present, can alter such things as ligand specificity and/or binding affinity, pH tolerance, catalytic rate (in the case of enzymes), etc. That said, unless one knows in advance what specific three-dimensional sub-structure (and amino acid residues) confers a particular biochemical function, overall global structural similarity between proteins does not allow biochemical function to be assigned with any level of confidence.

To date, several general approaches have been developed to analyze protein structure. Experimental methods such as X-ray crystallography and NMR spectroscopy allow the derivation in some cases of high resolution three-dimensional structural models (i.e., an atomic resolution of less than about 2.5 Å). While experimental methods can produce high quality structural models in some cases, it is difficult, if not impossible, to predict in advance which proteins can be induced to form crystals suitable for X-ray crystallographic studies. In general, only about 20% of proteins appear to be amenable for X-ray crystallography, and certain classes of proteins, for example, integral membrane proteins and other non-soluble proteins, can not generally be studied by X-ray crystallography. Similarly, NMR analysis is limited to only certain classes of proteins, namely proteins that are soluble and have molecular weights of about 30 kilodaltons or less. Moreover, the various experimental methods for developing protein structural models are extremely capital and labor intensive. This is reflected by the fact that, to date, worldwide, only a few thousand non-redundant high resolution three-dimensional structural models derived from X-ray crystallography or NMR spectroscopy have been deposited in such international repositories as the Protein Data Bank.

In addition to experimental methods such as X-ray crystallography and NMR spectroscopy, various computational methods have also been developed to build three-dimensional structural models for proteins, including exact models. Such techniques include threading, comparative modeling, and ab initio methods. Although scientists have yet to fully understand the complex nature of how a protein, in some cases comprising thousands of amino acid residues, folds in situ to assume a biologically active conformation, such computational methods frequently enable low resolution three-dimensional structural models to be produced for many different proteins. A major advantage in computationally generating three-dimensional model structures is speed. For example, whereas it typically takes weeks to months to obtain a single structural model by experimental methods, depending on the algorithm used, the number and processing speed of the computers used, etc. thousands or more structural models can be generated per day.

Regardless of the method used to derive a model structure for a protein, to determine biochemical function(s) from a structural model, it is still necessary to identify the particular sub-structure(s) that confers function. A pioneering invention in this regard is described in U.S. Ser. No. 09/322,067, filed May 27, 1999. Briefly, therein described are functional site descriptors that define spatial relationships between specific amino acid residues that confer specific biochemical functions on proteins, and methods of using such descriptors to analyze protein structural models (even those that are inexact or of moderate to low resolution) to determine biochemical function.

Despite such inventions, the proteomics field is still in its infancy, and key to its advancement will be new inventions that further allow for truly high throughput analysis of large numbers of proteins to determine biochemical function and identify associated sub-structures. The instant invention represents a significant advance in this regard.

3. Definitions

Before describing the invention in general and in terms of specific embodiments, certain terms used in the context of the describing the invention will be defined. The following terms have the following meanings when used herein and in the appended claims. Those terms that are not defined below or elsewhere in the specification shall have their art-recognized meaning.

An “agonist” is a compound that binds to modulates the biochemical activity of a functional site of a protein. An agonist can be a “negative agonist,” i.e., a compound that decreases the activity of a protein, or a “positive agonist”, i.e., a compound that increases the activity of a protein. An “antagonist” is a compound that competes with another compound in interactions with a protein functional site. Agonists and antagonists include small molecules, proteins, lipids, and carbohydrates.

An “amino acid” is a molecule having the structure wherein a central carbon atom (the “alpha (α)-carbon atom”) is linked to a hydrogen atom, a carboxylic acid group (the carbon atom of which is referred to as a “carboxyl carbon atom”), an amino group (the nitrogen atom of which is referred to as an “amino nitrogen atom”), and a side chain group, R. In the process of being incorporated into a protein, an amino acid loses one or more atoms of its amino and carboxylic groups in a dehydration reaction that links one amino acid to another. As a result, when incorporated into a protein, an amino acid is often referred to as an “amino acid residue.” An amino acid may be derivatized or modified before or after incorporation into a protein (e.g., by glycosylation, by formation of cystine through the oxidation of the thiol side chains of two non-contiguous cysteine amino acid residues, resulting in a disulfide covalent bond that frequently plays an important role in stabilizing the folded conformation of a protein, etc.). An amino acid may be one that occurs in nature in proteins, or it may be non-naturally occurring (i.e., is produced by synthetic methods such as solid state and other automated synthesis methods). Examples of non-naturally occurring amino acids include α-amino isobutyric acid, 4-amino butyric acid, L-amino butyric acid, 6-amino hexanoic acid, 2-amino isobutyric acid, 3-amino propionic acid, ornithine, norlensine, norvaline, hydroxproline, sarcosine, citralline, cysteic acid, t-butylglyine, t-butylalanine, phenylylycine, cyclohexylalanine, β-alanine, fluoro-amino acids, designer amino acids (e.g., β-methyl amino acids, ac-methyl amino acids, Nα-methyl amino acids), and amino acid analogs in general.

In addition to its substitutent groups, two different enantiomeric forms of each amino acid exist, designated D and L. In mammals, only L-amino acids are incorporated into naturally occurring proteins, although the invention contemplates proteins incorporating one or more D- and L-amino acids, as well as proteins comprised of just D- or L-amino acid residues.

Herein, the following abbreviations may be used for the following amino acids (and residues thereof): alanine (Ala, A); arginine (Arg, R); asparagine (Asn, N); aspartic acid (Asp, D); cyteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

As will be appreciated, many embodiments of the invention are implemented in silico. In such embodiments, actual physically existing amino acids, peptide fragments, etc. are not employed; instead, electronic or other machine manipulable data forms representing these molecules are used. It is understood that in such embodiments, the foregoing nomenclature, while preferable, need not be used. Instead, any suitable nomenclature for such data forms may be employed.

It will also be appreciated that in the context of a consensus functional site profile, if desired, an amino acid residue may be represented as a variable when more than one amino acid residue (including no amino acid residue) is to be represented at a given position. In such cases, it is preferred that the variable be defined in terms of which amino acids (including no amino acid) may be present at the particular position, although the invention also envisions, but does not require, the inclusion of one or more user-defined “wild cards” in a functional site profile. A “wild card” may be defined in any manner, ranging from allowance of any (or no) amino acid at the particular position to a subset of as few as two different amino acids (or single specific amino acid and no amino acid).

A “β-carbon atom” refers to the carbon atom (if present) in the R group of the side chain of an amino acid (residue) that is covalently bonded to the α-carbon atom of that amino acid (residue).

By “comparison value” is meant a numerical value assigned to a degree of similarity based on a comparison between at least two amino acids in at least two proteins. Generally, comparison values are obtained by comparing amino acids of one functional site profile with one or more other functional site profiles.

Two amino acids are “contiguous” when they are linked by a peptide bond in a protein, or when one immediately precedes or follows the other in a representation of a protein, as in, for example, the amino acid sequence “A-R-T”. In this example, A and R are contiguous, as are R and T. While a fragment (or a corresponding representation thereof) containing contiguous amino acid residues can be of any length, typically they contain less than about 100 amino acids, preferably less than about 50 amino acids, more preferably less than about 25, and even more preferably about 15 or fewer (e.g., 14, 13, 12, 11, 10, 9, 8, 7, 6, 5, 4, 3, and 2) contiguous amino acids.

An “experimental method” refers to a method of determining information empirically, for example, via biochemical or biophysical assay or evaluation of the three-dimensional structure of a protein to atomic level resolution using a technique such as x-ray crystallography or NMR spectroscopy.

A “functional site” of a protein refers to any site in a protein that has a function. Representative examples include active sites (i.e., those sites in catalytic proteins where catalysis occurs), protein-protein interaction sites, sites for chemical modification (e.g., glycosylation and phosphorylation sites), and ligand binding sites. Ligand binding sites include metal ion binding sites, co-factor binding sites, antigen binding sites, substrate channels and tunnels, and substrate binding sites. In an enzyme, a ligand binding site that is a substrate binding site may also be an active site, or overlap with an active site. As used herein, the “biochemical function” of a functional site refers to the function carried out by the site in a naturally occurring protein that possesses the corresponding function. For example, the biochemical function of an active site refers to the specific catalytic activity of the site, whereas the biochemical function of a substrate binding site is the binding of the particular substrate.

In the context of the invention, a “functional family” refers to a family of proteins that exhibit the same function, e.g., two proteins that exhibit protein tyrosine kinase activity are members of the same family, or otherwise possess a common functional attribute that allows for classification. A “functional sub-family” refers to members of a functional family that share at least one or more additional functional attributes, e.g., in addition to exhibiting protein tyrosine kinase activity, each protein also acts on the same substrate) that also allow for classification based on those attributes.

A “functional site descriptor” refers to a minimal descriptor for defining the spatial configuration of a protein functional site that corresponds to a biological function. Preferred functional site descriptors are described in U.S. patent application Ser. Nos. 09/322,067 and 09/839,821.

A “functional site profile” is a representation of a protein functional site that associates (i.e., brings together in a meaningful way) representations of two or more non-contiguous amino acid residues, peptide fragments, or a combination of at least one peptide fragment and at least one amino acid residue, in the spatial environment of the functional site.

By “high throughput method” is meant capable of processing multiple sequences, for example.

By “in silico” is meant through use of a computer.

The term “modulate” refers to a change in the biochemical activity corresponding to the functional site profile. For example, modulation may involve an increase or a decrease in catalytic rate, substrate binding characteristics, etc. Modulation may occur by covalent or non-covalent interaction with the protein, and can involve an increase or decrease in biochemical activity. A “modulator” refers to a compound that causes a change, i.e., an increase or decrease, in activity of a protein, and is typically a ligand, either peptidic, polypeptidic, or small molecule (e.g., an agonist or antagonist). A modulator may act directly, for example, by interacting with a protein to cause an increase or decrease in activity. A modulator may also act indirectly, for example, by interfering with, i.e., antagonizing or blocking, the action of another molecule that causes an increase or decrease in activity of the protein.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

A “plurality” means more than one.

A “polyhedron” means a geometric shape describing a volume.

In general, the term “protein” refers to any polymer of two or more individual amino acids (whether or not naturally occurring) linked via peptide bonds, as occur when the carboxyl carbon atom of the carboxylic acid group bonded to the α-carbon of one amino acid (or amino acid residue) becomes covalently bound to the amino nitrogen atom of the amino group bonded to the α-carbon of an adjacent amino acid. These peptide bond linkages, and the atoms comprising them (i.e., α-carbon atoms, carboxyl carbon atoms (and their substituent oxygen atoms), and amino nitrogen atoms (and their substituent hydrogen atoms)) form the “polypeptide backbone” of the protein. In simplest terms, the polypeptide backbone shall be understood to refer the amino nitrogen atoms, α-carbon atoms, and carboxyl carbon atoms of the protein, although two or more of these atoms (with or without their substituent atoms) may also be represented as a pseudoatom. Indeed, any representation representing a polypeptide backbone that can be used in the context of the invention will be understood to be included within the meaning of the term “polypeptide backbone.”

As used herein, the term “protein” refers to proteins that have one or more functional sites under physiological conditions (i.e., the conditions in which the protein is found in nature, or if not found in nature, the conditions under which the protein is ultimately intended to be used). In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated.

The term “derivative” refers to a chemical modification of a protein. A derivative protein, e.g., one modified by glycosylation, pegylation, or any similar process, retains the biochemical activity corresponding to the functional site profile.

The term “fragment” of a protein refers to a polypeptide comprising fewer than all of the amino acid residues of the naturally occurring or otherwise pre-existing or known protein but retains the biochemical activity corresponding to the functional site profile. As will be appreciated, a “fragment” of a protein may be a form of the protein truncated at the amino terminus, the carboxy terminus, and/or internally (such as by natural splicing), and may also be variant and/or derivative. A “domain” of a protein is also a fragment, and comprises the amino acid residues of the naturally occurring or otherwise pre-existing or known protein required to confer the biochemical activity corresponding to the functional site profile.

A “homologue” refers to a protein that has evolutionary relationship with another protein, i.e., the two proteins are descendents of a common ancestral protein.

A “variant” or “analog” refers to a protein altered by one or more amino acids in relation to a reference protein (e.g., a naturally occurring form of the protein), for example, by one or more amino acid sequence substitutions, deletions, and/or insertions. A variant may have “conservative” changes, wherein a substituted amino acid has similar structural or chemical properties (e.g., replacement of leucine with isoleucine). Alternatively, a variant may one or more have “non-conservative” changes (e.g., replacement of glycine with tryptophan). Other variations include amino acid deletions or insertions, or both.

Unless otherwise indicated, a protein's amino acid sequence (i.e., its “primary structure” or “primary sequence”) will be written from amino-terminus to carboxy-terminus. In non-biological systems (e.g., those employing solid state synthesis), the primary structure of a protein (which also includes disulfide (cysteine) bond locations) can be determined by the user. As a result, proteins having primary structures that duplicate those of biologically produced proteins can be achieved. In addition, completely novel proteins, or proteins containing one or more novel portions, can also be synthesized, as can proteins incorporating non-naturally occurring amino acids.

Similarly, a functional site profile will be written from amino-terminus to carboxy-terminus, unless otherwise indicated. Of course, if desired, one or more of the peptide fragments used to assemble a functional site profile may be re-ordered or written other than from amino-terminus to carboxy-terminus, although herein any such break with convention will be indicated.

In addition to primary structure, proteins also have secondary, tertiary, and, in multisubunit proteins, quaternary structure. “Secondary structure” refers to local conformation of the protein chain, with reference to the covalently linked atoms of the peptide bonds and α-carbon linkages that string the amino acid residues of the protein together. Representative examples of secondary structures include α helices, parallel and anti-parallel β structures, and structural motifs such as helix-turn-helix, β-α-β, the leucine zipper, the zinc finger, the β-barrel, and the immunoglobulin fold. “Tertiary structure” concerns the three-dimensional structure of a protein, including the spatial relationships of amino acid side chains and atoms, and the geometric relationships of different regions of the protein. “Quaternary structure” refers to the structure and non-covalent association of different polypeptide subunits in a multisubunit protein.

A “pharmacophore” refers to spatially oriented functional groups (i.e., chemical groups) that confer activity to a chemical compound at a target. An “inverse pharmacophore” refers to the spatially oriented components of a protein responsible for interacting with a chemical compound that modulates the protein's activity. In the context of pharmacophores, the term “complementary” refers to a correspondence between a pharmacophore and an inverse pharmacophore, such that constituents of a pharmacophore can interact with constituents of an inverse pharmacophore. For instance, a hydrogen bond donor in a ligand may constitute a portion of a pharmacophore that interacts with a complementary hydrogen bond acceptor in a protein that constitutes a portion of the inverse pharmacophore.

A “pseudoatom” refers to a representation of two or more atoms in a protein or amino acid. Representative examples of pseudoatoms include an amino acid side chain center of mass, a center of mass (or, alternatively, the average position) of an α-carbon atom and the carboxyl atom bonded thereto, and a center of mass of the α-carbon atoms of two adjacent (but not necessarily contiguous) amino acid residues. With regard to spatial relationships, as with representations of other atoms, a pseudoatom's position in three-dimensional space (represented typically by an x, y, and z coordinate set) represents the average (or weighted average) position of two or more atoms.

A “reduced model” refers to a three-dimensional structural model of a protein wherein fewer than all heavy atoms (e.g., carbon, oxygen, nitrogen, and sulfur atoms) of the protein are represented. For example, a reduced model might consist of representing just the positions of, for example, the α-carbon atoms; the carbon, nitrogen, and oxygen atoms of the polypeptide backbone; or pseudoatoms each representing all of the atoms for an individual amino acid residue of the protein, with each amino acid connected to the subsequent amino acid by a virtual bond. Other examples of reduced protein models include those in which only the α-carbon atoms and side chain centers of mass of each amino acid are represented, or preferably, where only the polypeptide backbone is represented.

Protein structures useful in the practice of the invention can be of different quality. The highest quality determination methods are experimental structure prediction methods based on x-ray crystallography and NMR spectroscopy. In x-ray crystallography, “high resolution” structures are those wherein atomic positions are determined at a resolution of about 2 Å or less, and enable the determination of the three-dimensional positioning of each atom (or each non-hydrogen atom) of a protein. “Medium resolution” structures are those wherein atomic positioning is determined at about the 2-4 Å level, while “low resolution” structures are those wherein the atomic positioning is determined in about the 4-8 Å range. Herein, protein structures that have been determined by x-ray crystallography or NMR may be referred to as “experimental structures,” as compared to those determined by computational methods, i.e., derived from the application of one or more computer algorithms to a primary amino acid sequence to predict protein structure.

As alluded to above, protein structures can also be determined entirely by computational methods, including, homology modeling, threading, and ab initio methods. Often, models produced by such computational methods are reduced models. Of course, it is understood that once a protein structure based on a reduced model has been generated, all or a portion of it may be further refined using any suitable method to include additional predicted detail, up to and including all atom positions.

Computational methods usually produce lower quality structural models than models derived from data collected from experimental methods such as X-ray crystallography and NMR spectroscopy. While not necessary to practice the instant methods, the precision of models generated by computational methods can be determined using a benchmark set of proteins whose structures are already known. The computationally-derived model for each protein may then be compared to a corresponding experimentally determined structure. The difference between the computationally-derived model and the experimentally determined structure can be quantified via a measure called “root mean square deviation” (RMSD). A model having an RMSD of about 2.0 Å or less as compared to a corresponding experimentally determined structure is considered a “high quality” or “high resolution” model. Frequently, computationally-derived models have an RMSD of about 2.0 Å to about 8.0 Å when compared to one or more experimentally determined structures, and are called “inexact models”. As with models derived from experimental data, “moderate resolution” computational models have an RMSD of about 2.0 Å to about 4.0 Å as compared to a corresponding experimentally determined structure, and “low resolution” computational models have an RMSD of about 4.0 Å to about 8.0 Å as compared to a corresponding experimentally determined structure. As will be appreciated, computational modeling techniques can also be used to generate model protein structures for which no corresponding experimentally derived structures are available. Such models are referred to as “approximate models”, as there is no experimental structure for comparison.

A “spatial environment” of a protein functional site refers to some volume encompassing those amino acid residues defined in a functional site descriptor responsible for conferring activity to the protein.

By “spatially local” is meant a local environment about a defined position within a protein three-dimensional structure (be it an experimental structure or a computational model) wherein the local environment is itself defined by any given measure (e.g., a radius) about the defined position in three dimensions.

The terms “specific binding”, “specifically binding”, “specificity”, and the like refer to an interaction between a protein and a modulator (e.g., an agonist or an antagonist), an antibody, etc., that is not random. “Selective binding”, “selectivity”, and the like refer the preference of a compound to interact with one molecule as compared to another. Preferably, interactions between compounds, particularly modulators, and proteins are both specific and selective.

A “target protein” refers to a protein used in a discovery process. In general, target proteins are used in screening assays to identify compounds that modulate the activity of the protein.

### SUMMARY OF THE INVENTION

The object of this invention is to provide functional site profiles, and methods and tools for creating and using functional site profiles, for protein functional sites that confer particular biochemical functions. Such profiles have numerous applications. For example, they can be used to determine if a protein has a particular biochemical function, and, if so, the amino acid residues present in the site; to classify proteins based on biochemical function, for example, into protein families based on biochemical function; to further classify protein families into subfamilies; to identify substrate or ligand binding specificity; and to produce structural models of functional sites corresponding to a functional site profile. Functional site profiles, and methods of using them, can also be used in the context of drug discovery, for example, in processes to identify compounds that specifically interact with specific proteins and to identify inverse pharmacophores that can be used to generate complementary pharmacophores that can then be used in screening compound libraries as well as in designing compounds for screening. The functional site profiles of the invention can also be used in conjunction with other function determination methods (e.g., those based on functional site descriptors), for example, to assess or confirm other information, to provide additional data, etc.

As will be appreciated, the functional site profiles of the present invention are suitable for high throughput applications and may be readily implemented by computers executing computer program logic embodying such methods. Additionally, it will be appreciated that the instant functional site profiles and methods can be implemented in software.

Thus, in one aspect, the invention concerns functional site profiles. Functional site profiles are representations of protein functional sites that associate representations of two or more non-contiguous amino acid residues, peptide fragments, or combinations of non-contiguous amino acid residues and non-contiguous peptide fragments that are found in the spatial environment of the functional site as depicted, for example, in a model of the protein, or a region of the protein that includes the functional site.

Functional site profiles can be developed for any class of protein functional sites. These classes include active sites (i.e., catalytic sites in enzymes), protein-protein interaction sites, sites for chemical modification (e.g., glycosylation and phosphorylation sites), and ligand binding sites (e.g., metal ion binding sites, co-factor binding sites, antigen binding sites, substrate channels and tunnels, and substrate binding sites).

Functional site profiles can be developed for any protein having one or more functional sites, and unique profiles can be developed for each site. The protein may be of eukaryotic, prokaryotic, archaeal, or viral origin. Prokaryotic organisms include bacteria. Eukaryotic organisms include plants and animals, particularly those of medical or agricultural import. A representative class is vertebrate animals, which includes mammals, fish, and birds. Preferred mammals include bovine, canine, equine, feline, ovine, porcine, and primate animals, as well as humans.

In certain preferred embodiments, protein models used in the practice of the invention are reduced models. The models may be prepared computationally by homology or comparative modeling, threading, ab initio or other computational methods. In alternative embodiments, the models may be prepared from data gathered by experimental methods such as x-ray crystallography or NMR spectroscopy. The models may be inexact models, or models of better quality, e.g., high resolution or moderate resolution models.

In certain preferred embodiments, the protein model represents a three-dimensional structure for the complete protein, i.e., all of the amino acid residues of the protein as it occurs in nature, for example. In other preferred embodiments, the protein model represents only a portion of the protein, for instance, a domain of a multi-domain protein, such as a domain that possesses the biochemical activity that corresponds to or contains the amino acid residues represented by the functional site profile.

In any event, the protein model provides a representation of the spatial environment of the functional site. Preferably, the representation is a volume that contains the amino acids that comprise the functional site. Typically, the volume will contain between 2 and about 300 amino acid residues. The volume can be defined by any geometric shape, or combination of geometric shapes. Preferably, the volume is defined by one or more polyhedron, preferably spheres. In preferred embodiments, the polyhedron(s) is centered about one or more of the amino acid residues known to be required for the particular biochemical function. The identity of such residues can be derived from any source, for example, the scientific literature, by experimental methods such as mutagenesis, or, preferably, through the use of a minimal functional site descriptor for the particular function that identifies a specific amino acid residue, or subset of amino acid residues, at a particular amino acid position in a protein. Alternatively, the volume(s) employed may be centered on other representations of the functional site (e.g., the center of the functional site, for example, as defined by a corresponding functional site descriptor) known to be involved in the biochemical function. When a sphere is employed, it is preferred to maximize its radius, but only to the extent that the radius employed does not result in the inclusion in the signature of amino acid residues that lead to the identification of false positives, i.e., proteins identified by the functional site profile as possessing the corresponding biochemical activity but which, in fact, do not possess that activity. It will be appreciated that when multiple polyhedrons are employed in the examination, they may be of different sizes (e.g., spheres having different radii), with the total volume defined by the polyhedrons being defined as the union of the volumes of each of the polyhedrons. In preferred embodiments when one or more spheres are used to define the volume representing a spatial environment, a radius of less than about 30 Å, preferably less than about 20 Å (e.g., 19, 18, 17, 16, 15, 14, 13, 12, 11, 10, 9, 8, 7, 6, and 5 Å), with a radius of about 10 Å being particularly preferred. In embodiments employing more than one sphere (or other volumetric shape, the union of the volumes of which comprises the spatial environment from which amino acid residues are identified for inclusion in a function site protile), equal or different radii may be employed with respect to each sphere (or other shape).

In preferred embodiments, the volume representing a spatial environment is centered on a representation of an amino acid residue, or an atom thereof (e.g., an atom of the amino acid (or pseudoatom representing two or more atoms) that forms a part of the polypeptide backbone of the protein), in the protein functional site. In certain preferred embodiments, an amino acid on which a volume representing a spatial environment is centered is an amino acid that is known to be involved in or essential to the activity of the functional site.

This examination of the spatial environment of the functional site results in the identification of peptide fragments, each of which may be comprised of two or more contiguous amino acid residues, alone or in conjunction with single amino acid residues, in the protein that are in the neighborhood, or spatial environment, of the functional site. The fragments (and single amino acid residues, if any) within the volume of the polyhedron(s) used for the analysis are then extracted and preferably combined in the order they appear in the protein from which they were extracted (amino-terminus to carboxy-terminus, beginning with the most amino-terminal fragment or amino acid and ending with the most carboxy-terminal fragment or amino acid) to create the particular functional site profile. As will be appreciated, consensus signatures for a given biochemical function can also be produced by developing independent signatures from a plurality of proteins having the same functional site. Each of these signatures can then be compared, and a consensus developed. When intended for computer implementation, it is preferred, but not required, that the functional site profiles be no larger than necessary to uniquely identify the corresponding site for in one or more proteins.

Consensus functional site profiles can also be created for families of proteins exhibiting similar biochemical functions. Typically, a consensus functional site profile is derived from an alignment of two or more functional site profiles identified from two or more proteins that exhibit a particular biochemical function.

Certain embodiments of the invention concern libraries comprised of a plurality of different functional site profiles. In some embodiments, each of the functional site profiles corresponds to a different biochemical function. Other embodiments concern libraries comprised of one or more functional site profiles that have been identified from proteins exhibiting similar biochemical functions, alone or in conjunction with one or more functional site profiles for one or more other functions. Typically, libraries of functional site profiles exist as databases that can be accessed by a computer or computer system.

In a related aspect, the invention concerns methods for creating functional site profiles for functional sites determined to confer particular biochemical functions on proteins that possess those functional sites. For a given functional site profile, in general these methods first involve obtaining a protein model that represents a plurality of amino acid residues in a spatial environment of a protein functional site and identifying in the protein model at least two non-contiguous amino acid residues, peptide fragments, or combinations of non-contiguous amino acid residues and non-contiguous peptide fragments in the plurality of amino acid residues in the spatial environment of the protein functional site. The representations of the non-contiguous amino acid residues and/or peptide fragment are then assembled to make the functional site profile.

In preferred embodiments of this aspect, one or more of each of the non-contiguous amino acid residues identified in the spatial environment is part of a peptide fragment (e.g., two or more amino acid residues) in the spatial environment. In such embodiments, some or all of the residues of the peptide fragment (all of which residues are in the spatial environment) may also be identified and extracted for inclusion in a functional site profile. After identifying amino acids, and preferably peptide fragments, in the spatial environment of the protein functional site, the various amino acids and peptide fragments (each of which may be referred to herein as a different “peptide element”) are assembled to make a functional site profile for a protein functional site that confers a particular biochemical function. The assembly of the amino acids and peptide fragments (or peptide elements) into a functional site profile can occur by any suitable method. In certain preferred embodiments, the amino acids and/or peptide fragments are arrayed contiguously, i.e., end to end, preferably in the order they appear in an amino acid sequence of the protein.

Other embodiments of this aspect relate to methods for making consensus functional site profiles. Generally, a consensus functional site profile is made by first creating an independent functional site profile for each of a plurality of different proteins comprising different amino acid sequences but exhibiting similar or related biochemical functions. The consensus functional site profile is then developed by comparing two or more of the independent functional site profiles. In preferred embodiments, the comparison is performed by aligning the sequences to be compared, e.g., preferably using an automated multiple sequence alignment tool.

Because the various aspects and embodiments of the invention are amenable to computer-based automation, e.g., by implementing the functional site profiles and related methods using computer programs comprising computer program logic configured to direct a processor to perform the requisite functions, computer program products comprising the same represent another aspect of the invention. Certain embodiments of this aspect thus relate to computer program products comprising a computer useable medium storing data that represents one or more functional site profiles of the invention. When a plurality of functional site profiles are stored (i.e., a functional site profile library), the functional site profiles in the library preferably are derived from different proteins, although the invention includes embodiments where more than one functional site profile is created for a given protein. For example, the functional site profiles may each represent only a subset of the amino acids and/or peptide fragments in a given spatial environment, although in preferred embodiments. Alternatively, some or all of the functional site profiles for a given protein and its functional site may be developed using different functional site spatial environments. When the functional site profiles are derived from different proteins, the proteins may be of the same functional family, or from different functional families. Functional site profiles for functional sub-families may also be included, as can consensus functional site profiles.

Other embodiments of this aspect concern computer program products comprising a computer useable medium that stores computer program logic configured to direct a processor to execute instructions that implement the methods of the invention.

Another aspect of the invention concerns the use of a functional site profile to assign a biochemical function a protein of unknown biochemical function. Here, a “protein of unknown biochemical function” includes proteins for which no biochemical function is known, as well as proteins of already known, but different biochemical function as compared to the biochemical function represented by the signature.

In certain embodiments of this aspect, the functional site profile from a protein known to exhibit a specific biochemical function is compared to an amino acid sequence, up to and including the complete amino acid sequence, of a protein of interest. If a portion of the query protein's sequence is determined to contain the functional site profile for the biochemical function, for example, by alignment, the query protein is assigned as having the biochemical function that corresponds to the functional site profile from the protein known to have the biochemical function. In embodiments when the functional site profile is aligned with the amino acid sequence of the protein of interest, it is preferred to use a scoring function that evaluates if the alignment between the portion of the amino acid sequence of the protein and the functional site profile is indicative of the existence of the functional site profile in the protein.

As with other aspects and embodiments of the invention, this aspect and its embodiments are preferably implemented in an automated manner, e.g., through the use of a computer system. In addition, as with the other methods of the invention, these methods may be applied to a plurality of proteins of interest, pluralities of functional site profiles can be used, including the use of multiple functional site profiles for the same or different biochemical functions.

In other embodiments of this aspect, the functional site profile from the protein known to have the biochemical function is compared to a set of peptide sequences from a protein of interest that have been assembled to form a query functional site profile. In these embodiments, the set of peptide sequences preferably is spatially local to at least one amino acid residue of the protein of interest that corresponds to a pre-selected amino acid residue (preferably, two or more residues) from the known protein, e.g., an amino acid known to be required in order for a protein to have a particular biochemical function. This second group of sequences (or a combination of individual amino acids and peptide sequences) is preferably arranged contiguously in order of their occurrence in the query protein to form the query functional site profile. If the signatures are similar, the biochemical function of the protein known to have the biochemical function can be assigned to the query protein.

Functional site profiles can also be used to classify proteins. Typically, such classification is based on biochemical function. In general, the methods of this aspect involve obtaining an amino acid sequence for one or more proteins of interest and analyzing the sequences with one or more functional site profiles to determine if the functional site profile(s) exists in the amino acid sequence of the protein of interest. If so, the protein of interest is classified as having the biochemical function corresponding to the functional site profile. Representative classifications include family and sub-family classifications.

These and other functional site profile-based classifications can be used for many purposes, for example, in drug discovery. As will be appreciated, the methods of the invention allow proteins having specific biochemical functions to be identified. Additionally, the relevant portions, e.g., amino acids, peptide fragments, etc., in and around a functional site can be identified. In preferred embodiments, an amino acid sequence for a protein of interest is obtained and analyzed with a functional site profile for a particular biochemical function to determine if the functional site profile exists in the amino acid sequence of the protein of interest. If the functional site profile is found, the amino acid residues in the protein of interest that correspond to the functional site profile can be identified, thereby identifying the functional site. If desired, the three-dimensional structure of the functional site can then be determined by experimental or computational methods.

The information about a functional site (e.g., the identities of the amino acids within a user-defined spatial environment of the site, the positions of such amino acid residues in three-dimensional space, etc.) identified by or corresponding to a functional sire profile can be used, for example, to identify one or more compound that specifically interacts with the functional site of the target protein. In addition to providing specificity in interactions between the functional site of a protein (or more than one protein, e.g., one more proteins in same family as the protein of interest) and a compound, selectivity can also be addressed by identifying functional sites, and functional site profiles, corresponding to the functional site profile in other members of a target protein's protein family. In particular, the differences between the functional site profiles of proteins within the same family or sub-family may be used to enhance the selectivity of binding by one or more compounds to a protein of interest.

Still another aspect of the invention concerns method of creating inverse pharmacophores based on functional site profiles. In general, such methods involve identifying amino acid residues that comprise a functional site in a target protein through the use of one or more functional site profiles and then obtaining a three-dimensional structural representation of the functional site, for example, by an experimental method (e.g., x-ray crystallography or NMR spectroscopy) or a computational method (e.g., homology modeling, threading, or ab initio), or by a combination of experimental and computational methods. Some or all of the foregoing information may then be used to develop one or more inverse pharmacophores for the functional site.

Closely related to the inverse pharmacophore aspect of the invention are methods for developing pharmacophores complementary thereto, as well as identifying compounds that match criteria established by the pharmacophore. These aspects and embodiments are also preferably implemented via a computer.

An aspect related to methods for identifying compounds, pharmacophores, inverse pharmacophores, and the like concerns compounds that are identified through the use of such methods, and compositions comprising the same.

Yet another aspect of the invention concerns methods for confirming structure-based assignments of biochemical function, preferably those made using a functional site descriptor that describes a functional site. Typically, such methods comprise developing a functional site profile for a protein previously determined to have the particular biochemical function. As with other functional profiles of the invention, such profiles preferably contain more, or alternative, information than the corresponding functional site descriptor. In preferred embodiments, the signature is developed using a model structure of the protein the function of which is desired to be confirmed. By examining the three dimensional space (e.g., the spatial environment) in and around that portion of the protein previously determined to confer the particular biochemical function (i.e., the spatial environment of the functional site), a plurality of amino acid residues, preferably between 5 to 50 or more amino acid residues involved in the particular biochemical function, can be identified. Such examination preferably involves the use of computational techniques to identify amino acid residues within a certain distance of, e.g., spatially local to, the amino acid residue known to be involved in the function. Those amino acids, peptide fragments, and the like identified in the spatial environment can then be used to generate a functional site profile for the functional site. That profile can then be compared with a functional site profile for a protein determined to have the particular function by method another. If the comparison reveals that the functional site profiles are sufficiently similar to be indicative of the same biochemical function, the previous function assignment is deemed to be confirmed.

In preferred embodiments of this aspect of the invention, when a functional site profile from a protein known to have the particular biochemical function is to be used to confirm a structure-based function assignment, an analogous process may be followed for the query protein. Specifically, a functional site profile is developed for the functional site of the query protein. The signature from the query protein, or “protein of interest”, may then be compared with the signature from the known protein. If the signatures from the known and query proteins are similar, the biochemical function assignment for the query protein is confirmed. Any suitable scoring metric, or combination of metrics, can be used to assess the similarity between the signatures. As will be appreciated, assessing similarity between profiles can be applied with respect to any aspect or embodiment of the invention, as desired.

In preferred embodiments, the comparison is performed via a sequence alignment, where amino acid residue variability (or conservation) at each amino acid residue position is compared and scored. The cumulated signature score thus calculated for the query protein can then be assessed to determine whether it goes beyond a pre-selected threshold indicative of confirmation of function. Again, any suitable scoring metric, or combination of metrics, can be used for such an assessment.

As those in the art will appreciate, the methods of the invention can be implemented in an automated fashion, preferably by a computer. Such automated methods, computer program products comprising computer program code logic stored in a computer useable medium, and computer systems used to implement them, represent additional aspects of the invention. The methods of the present invention can be described as a plurality of instructions being performed by a data processor, such that the methods can be implemented in hardware, software, firmware, or a combination thereof. As software, or computer program products, the methods can be implemented in any suitable programming language that is compatible with the computer hardware and operating system which is performing the instructions, and can be selected and adapted by one of ordinary skill in the art. Examples of such suitable programming languages include, but are not limited to, Fortran, C, and C++.

Also, after development of one or more functional site profiles (for the same or different biochemical functions) from proteins known to have certain structure-based biochemical function, the signatures (or a library comprising a plurality of such signatures) can be used in one-dimensional sequence analyses, without the need to produce structures, experimentally or computationally, for query proteins. As such, these embodiments, as well as the other aspects and embodiments of the invention, are amenable to computerization, and can be used to rapidly analyze multiple amino acid sequences, even on proteome-wide scales.

### BRIEF DESCRIPTION OF THE FIGURES

FIGS. 1(A) and (B) two representative embodiments of spatial environments about a protein's active site. In 1(A), the spatial environment is depicted as a single sphere centered about the center of a functional site initially identified by a functional site descriptor. As shown, the volume defined by the sphere contains sub-structures comprising subsets of contiguous amino acids (i. e., peptide fragments) within the protein. In 1(B), the spatial environment is defined for the same protein using three spheres, each centered about an amino acid residue identified by the functional site descriptor used to identify the functional site. Together, the volume defined by the overlapping spheres represents the spatial environment of the functional site. As indicated, amino acids and peptide fragments resident inside the spatial envorinment the differ somewhat from those in 1(A).

FIG. 2 lists 193 FFFs used to describe active sites that carry out a wide range of enzymatic activities and represent functions across all six Enzyme Commission (EC) classes.

FIG. 3 illustrates in three parts, (A), (B), and (C), the construction of a functional site profile of the invention. 3(A) illustrates a tertiary structure for the protein 1ivyA, a member of the serine carboxypeptidase family, as derived from the PDB. The active site was located using a functional site descriptor that that defined the active site based on the identity and three-dimensional relationship of three key amino acid residues (highlighted spheres). Peptide fragments (highlighted) were located using a spatial environment defined with spheres having 10 Å radii, with each sphere being centered on one of the three residues of the functional site descriptor. Note that the volume defined by the spatial environment is not illustrated. 3(B) is a magnified view of the six non-contiguous peptide fragments defined by the spatial environment,with the portions of the protein outside the spatial environment not being shown. The amino acid sequence for each of the extracted fragment appears in the top line of 3(C). The second line of 3(C) shows a preferred embodiment of the invention, namely a functional site profile for the catalytic site of protein 1ivyA assembled as a single contiguous profile sequence by combining the peptide fragments in the order they appear in the protein (amino-terminus to carboxy-terminus, beginning with the most amino-terminal fragment or amino acid and ending with the most carboxy-terminal fragment or amino acid).

FIG. 4 has two parts, (A) and (B). 4(A) shows an alignment of functional site profiles for the active sites in five members of the serine carboxypeptidase functional family (PDB codes 1ysc,1ivyA, 1ivyB, 1ac5, 1cpy). The ClustalW alignment score is shown beneath each residue column (“*”=identical or conserved residues in all sequences in the alignment; “:”=conserved substitutions; “.”=semi-conserved substitutions). Note that some regions of the functional site profiles are highly conserved across the family, while other regions show significant variation. These areas of conservation and variation can be exploited to design or identify specific and selective compounds for the modulation of serine carboxypeptidase activity. 4(B) shows another alignment of functional site profiles, including the same five functional site profiles for the serine carboxypeptidase active site as shown in 4(A), plus the nearest active site structure decoy, 2-hydroxy-6-oxo-6-phenylhexa-2,4-dienoate hydrolase (PDB code 1c4x). FIG. 5 shows two graphs, 5(A) and 5(B). 5(A) plots the family functional site profile scores for true active sites versus non-homologous decoy active sites. A difference in score of at least 0.25 between true and decoy sites is seen for 158 of the 170 functions for which the closest decoy site is from a protein that is not homologous to the proteins containing true active sites. 5(B) plots family functional site profile scores for true active sites versus decoy sites for 23 functions for which the closest decoy site is from a protein that is homologous to one or more proteins containing the true active site. The overall homology has little affect on local functional site profiling, as only three of the functions have a difference in score of less than 0.25. The 23 functions in this category are: 1,3,8-trihydroxynaphthalene reductase catalytic site, 3alpha, 20beta-hydroxysteroid dehydrogenase catalytic site, 7alpha-hydroxysteroid dehydrogenase catalytic site, ATP-dependent phosphoenolpyruvate carboxykinase catalytic site, bacterial lipase catalytic site, c-Jun N-terminal kinase catalytic site, calcium/calmodulin-dependent kinase catalytic site, cAMP-dependent kinase or phosphorylase kinase catalytic site (closed form), carbonyl reductase catalytic site, carboxylesterase catalytic site, chinese hamster ovary reductase and FR-1 reductase catalytic site, cis-biphenyl-2,3-dihydrodiol-2,3-dehydrogenase catalytic site, cyclodextrin glycosyltransferase catalytic site, cytochrome P450eryF monooxygenase catalytic site, exfoliative toxin A or B serine protease catalytic site, fungal lipase catalytic site, gelatinase-A catalytic site, N6 adenine-specific DNA methyltransferase catalytic site (gamma group), phosphoglycerate mutase catalytic site, prolyl oligopeptidase family catalytic site, Ras or Ran GTPase catalytic site, titin kinase catalytic site, and tonin catalytic site (inactive form).

FIG. 6 has two parts, (A) and (B). In 6(A), scores for pairwise alignments between different functional sites demonstrate the uniqueness of the functional site profiles. On average, scoring a site against a functionally unrelated one leads to a score of 0.054. From the ensemble of these non-equivalent site scores, discrimination was observed to be high, with the self-recognition score of 1.0 falling an average 21.9 standard deviations from the mean of the site mismatch. 6(B) shows score distributions for four distinct functions (each in a different panel), illustrating that functional site profile uniqueness varies according to the specific function.

FIG. 7 shows pairwise scores for consensus annotated genomic sequences. The pairwise scores are plotted for the 716 sequences that were assigned the same function by five tools: Blocks; BLAST; Pfam; PRINTS; and an FFF-based annotation method. 694 of the 716 sequences have functional sites that score at or above the twilight zone threshold of 0.25.

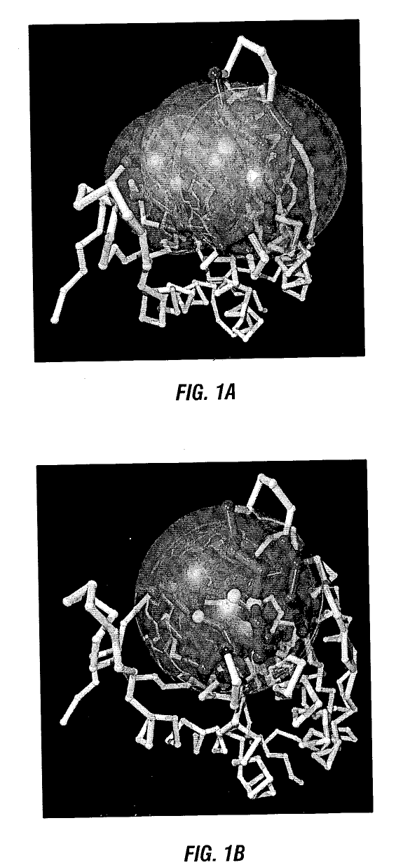

These and other aspects of the present invention will become evident upon reference to the following detailed description and attached drawings and sequence listing. In addition, various references are set forth which describe in more detail certain procedures and compositions.

### DETAILED DESCRIPTION

As is understood by one of skill in the art, structurally homologous protein families likely exhibit similar biochemical functions due to a conservation of active site chemistry and geometry. Although such functional sites are well conserved within families, a subset of key amino acid residues typically varies among the constituted proteins, and this differentiation results in their distinct biochemical activities (e.g., catalytic rate, substrate specificity, etc.). These detailed differences among family members allow precise recognition processes, and knowledge of them may be exploited in computational methodologies aimed at discovery of structural moieties involved in highly specific functions, as well as for other uses, such as in protein family and sub-family classification, protein engineering, and discovery of compounds that react specifically with a particular member (e.g.,a target protein), or subset of members, of a protein family. As those in the art will appreciate, by designing compounds that are both specific and selective for a target protein, problems that are often encountered later in the discovery process, e.g., toxicity (as may be caused by inadequate selectivity in binding, which can lead to undesired cross-reactivity with other members of the target protein's functional family or sub-family), can be avoided. In the field of pharmaceutical development, this can both shorten the discovery process and reduce the risk of failure during later stages, such as pre-clinical development during clinical trials.

The present invention is able to exploit differences that exist between proteins, even between proteins that differ in amino acid sequence by only one or a few amino acids, particularly if the differences exist in a protein functional site. This exploitation is possible because of the functional site profiles of the instant invention. A functional site profile is a representation of a protein functional site that associates (i.e., brings together in a meaningful way) representations of two or more non-contiguous amino acid residues, peptide fragments, or a combination of at least one peptide fragment and at least one amino acid residue, in the spatial environment of the functional site.

Functional site profiles are created by first producing a model of the three-dimensional structure of a protein, or at least a portion thereof that represents a spatial environment of a functional site. At least two non-contiguous peptide elements (where each peptide element is an amino acid residue or a peptide fragment within the spatial environment) are identified. These peptide elements are then assembled into a functional site profile. A functional site profile for a particular functional site in a particular protein will often be unique, as compared to other functional site profiles for analogous functional sites in other proteins. Indeed, it is preferred that that a functional site profile for a given functional site in a protein be unique. Such uniqueness is preferably imparted by selecting a spatial environment that allows a sufficient number of non-contiguous peptide elements and/or peptide elements of sufficient size, to be selected.

In order to generate a functional site profile according to the invention, a functional site to which the functional site descriptor will correspond must first be identified in a protein that is determined to have the function. In preferred embodiments, the functional site is identified using a functional site descriptor. Functional site descriptors are minimal descriptors that define the spatial configuration of a protein functional site that corresponds to a specific biological function. Preferred functional site descriptors, known in the art as “Fuzzy Functional Forms” of “FFFs” are described in U.S. patent application Ser. Nos. 09/322,067 and 09/839,821.

Functional site descriptors are used to identify functional sites in models of the three-dimensional structures of proteins. The models may be derived in any suitable manner, including an experimental method such as x-ray crystallography or NMR spectroscopy or a computational method such as homology modeling, threading, or ab initio folding. Models may be of any resolution or quality sufficient to be probed with a functional site descriptor. As will appreciated, the resolution or quality of the model required will, at least in part, be dictated by the requirements of the functional site descriptor utilized. In preferred embodiments, it is desired that the functional site descriptor allow for models of moderation to low resolution or quality to be employed, for example, such as the approximate or inexact models that may be produced in the course of genome-wide, computationally performed analyzes of protein structure. It will be appreciated that functional site descriptors that can be used in such circumstances will also be well suited for use in analyzing models of better quality.

After a functional site descriptor identifies the functional site in a protein model, a spatial environment for the functional site may be defined. A spatial environment is a volume encompassing those amino acid residues responsible for conferring the particular biological activity that corresponds to the functional site descriptor to the protein. Different volumes can be used to identify different spatial environments for a functional site. Volumes are typically defined by one or more polyhedrons, preferably spheres, centered on one or more features of the functional site. Particularly preferred features for centering polyhedrons are amino acids used in the functional site descriptor employed to identify the functional site. When multiple polyhedrons are employed to define a spatial environment for a functional site, it is preferred that the polyhedrons overlap, in which the volume representing the spatial environment in the included volume of the overlapping polyhedrons, which volume is also referred to herein as the union of the volumes of multiple polyhedrons.

The spatial environment defines a volume inside of which peptide elements can be identified. Initially, peptide elements are likely to be identified as representations of individual amino acids or peptide fragments protruding into the spatial environment. Of course, the information in the peptide fragments e.g., amino acid identities and positions, relative position in relation to other amino acids of the protein, spatially and/or in terms of sequence order, etc., may be represented by any system of nomenclature, provided that the nomenclature used can be translated into amino acid identity and positional information, if and when desired.

As a protein is a linear string of amino acid residues connected by peptide bonds, when it assumes a three dimensional structure, non-local residues (i.e., those adjacent to or near each other in the linear amino acid sequence) are brought into proximity. In a spatial environment, this results in non-contiguous peptide elements that protrude or extend into the defined volume.

To make a functional site profile for a particular functional site that confers a particular function on a protein possessing the site, representation for at least two non-contiguous peptide elements in the spatial environment are assembled, for example, into a sequence. Preferably, the representations for the non-contiguous peptide elements are assembled end to end, in the order they appear in the linear amino acid sequence. It will also be appreciated that for peptide elements that are peptide fragments, i.e., structures that comprise at least two amino acids or (representations thereof), it is possible, although not preferred, to extract only a portion of the residues in the fragment for inclusion in the functional site profile. If, for example, it was decided only to use four amino acids from a five amino acid peptide fragment, depending on which amino acid was not included, one or two different peptide fragments would result. Clearly, other permutations of this sort are possible and within the scope of the invention.

It is also understood that multiple functional site profiles can be developed for the same functional site. For example, different functional site profiles for the same function site in a protein can be created by using different spatial environments. For example, the spatial environment for one functional site profile may be defined as single sphere centered between two specific amino acid residues and having a radius of 10 Å, while the spatial environment for another functional site profile may be defined as single sphere centered between the same two specific amino acid residues but having a radius of 15 Å. Alternatively, spatial environments of equivalent volumes may be employed, but the volumes may be centered at different positions.

Structural Determination

Models of protein structure for use in conjunction with the invention can be determined by any suitable method. As will be appreciated, such models are used for identifying the amino acid residues in the spatial environment of a protein functional site. Representative examples of several experimental and computational methods useful in this regard are described below.

A. Experimental Analyses of Protein Structure

Protein structure can be assessed experimentally by any method capable of producing at least low resolution structural models. Such methods currently include x-ray crystallography (XRD) and nuclear magnetic resonance (NMR) spectroscopy. Models of protein structure elucidated by these methods are of varying quality. In certain embodiments of the invention, the production of functional site profiles utilizes high resolution or high quality structural models are desirable, although models of moderate or low resolution can also be used. To date, more than 2,000 non-redundant protein crystal structures have been solved. Data for these structures is available from a variety of sources, including the Protein Data Bank (PDB; Berman, et al. (2000), The Protein Data Bank. Nucleic Acids Research, vol. 28: 235-242).

Other techniques useful in studying protein structure include circular dichroism (CD), fluorescence, and ultraviolet-visible absorbance spectroscopy. See Physical Biochemistry: Applications to Biochemistry and Molecular Biology, 2^{nd} ed., W. H. Freeman & Co., New York, N.Y., 1982 for descriptions of these techniques. Such methods currently do not provide atomic level structural detail about proteins.

(i) X-Ray Crystallography (XRD)

X-ray crystallography, also referred to as x-ray diffraction (XRD) is one method for protein structural determination, and is based on the diffraction of X-ray radiation of a characteristic wavelength by electron clouds surrounding the atomic nuclei in the crystal. XRD uses crystals of purified proteins (but these frequently include solvent components, co-factors, substrates, or other ligands) to determine near atomic resolution the positions in three-dimensional space of the atoms making up the particular protein. Techniques for crystal growth are known in the art, and typically vary from protein to protein. Automated crystal growth techniques are also known.

Small molecules, i.e., those having a molecular weight of less than about 2,000 daltons (D), typically crystallize with fewer than several (frequently two) solvent components, with the atoms of the small molecule occupying a large majority, even greater than 90%, of the crystal volume. However, proteins are typically much larger (typically having molecular weights of 5,000-200,000 D), and when packaged into crystal lattice points, leave much larger gaps for inclusion of other molecules in the crystal. Thus, protein crystals typically contain 40-60% solvent. As a result, protein crystals have dynamic flexibility that can cause disorder in XRD studies and allow an observed electron density to be matched by more than one local conformation. Dynamic disorder can be reduced or eliminated by lowering the environmental temperature of the crystal during X-ray bombardment. Remaining static disorder may be due to one or more rigid static molecular conformations.

Detection of diffracted radiation enables the use of mathematical equations (e.g., Fourier synthesis) to generate three-dimensional electron density maps of the diffracted protein. Often, multiple reflections are required to make such determinations, with the number of reflections correlating positively with the resolution desired. Low numbers of reflections typically do not provide the requisite information to determine atomic positioning, although the position of a polypeptide chain (i.e., the chain trace) in individual protein molecules can often be fitted to the electron density map. Models resulting from these types of crystallographic data are often termed low resolution structural models. The fitting of a protein's amino acid sequence (for example, the primary structure of a protein solved by deducing the amino acid sequence encoded by a nucleic acid (e.g., a cDNA sequence) encoding the protein) to the determined electron density pattern allows the model of the protein's structure to be refined. Larger numbers of reflections and/or increasing refinement produces a higher resolution model of the protein structure.

It is important to note that while techniques such as XRD provide substantial information about protein structure, to date they provide only limited information about mechanisms of action. For XRD, this is due to the fact that the resulting models depict time-averaged atomic coordinates of atoms, and atoms undergo rapid dynamic fluctuation in solution, which can be important for the function of the protein. Indeed, on average the atoms in a protein are believed to oscillate over 0.7 Å per picosecond.

ii. Nuclear Magnetic Resonance (NMR) Spectroscopy

Nuclear magnetic resonance (NMR) spectroscopy enables determination of the solution conformation (rather than crystal structure) of proteins. Typically only small molecules, for example proteins of less that about 300 amino acids, are amenable to these techniques. However, recent advances have lead to the experimental elucidation of the solution structures of larger proteins, using such techniques as isotopic labeling. The advantage of NMR spectroscopy over x-ray crystallography is that the structure is determined in solution, rather than in a crystal lattice, where lattice neighbor interactions can alter the protein structure. Thus, dynamic motion of the protein in some time frames can be visualized by NMR spectroscopy. A disadvantage of NMR spectroscopy is that protein models derived from NMR data often is not as detailed or as well-resolved as a model generated by XRD.

Briefly, NMR spectroscopy uses radio frequency radiation to examine the environment of magnetic atomic nuclei in a homogeneous magnetic field pulsed with a specific radio frequency. These pulses perturb the nuclear magnetization of those atoms with nuclei of nonzero spin. Transient time domain signals are detected as the system returns to equilibrium. Fourier transformation of the transient signal into a frequency domain yields a one-dimensional NMR spectrum. Peaks in these spectra represent chemical shifts of the various active nuclei. The chemical shift of an atom is determined by its local electronic environment. Two-dimensional NMR experiments can provide information about the proximity of various atoms in the structure and in three dimensional space.

Protein structures can be determined by performing a number of two- (and sometimes 3- or 4-) dimensional NMR experiments on isotopically labeled protein and using the resulting information as constraints in a series of protein folding simulations. See Protein NMR Spectroscopy, Principles and Practice, J. Cavanagh, et al., Academic Press, San Diego, 1996, for a discussion of the many techniques associated with NMR spectroscopy.

iii. Conclusion

As described herein, experimentally solved protein structural models, particularly those solved to high resolution, can be used to in the creation of functional site profiles according to the invention. As the number of experimentally solved protein structural increases over time, the generation of new functional site profiles, or the modification of then-existing functional site profiles (if appropriate or necessary) will be facilitated. In addition, as the number of non-redundant protein structures increases, those structures will provide substrates that can be used in conjunction with various computational methods (e.g., homology modeling) for building protein models.

B. Methods for Producing Computationally-Derived Models of Protein Structure

While certain preferred embodiments of the invention that concern the production of functional site profiles involve the extraction of information about amino acid residues in the spatial environment of protein functional site from one or more experimentally solved structures, in other embodiments, the protein models from which the profiles are derived are computationally derived. Indeed, inexact or approximate models produced by a computational method (representative examples of which are described in greater detail below, or which are later developed) can be used. Of course, exact models and experimentally solved structures (particularly high and medium resolution structures) can also be used for such purposes.

i. Homology Modeling Techniques

Some methods for computationally deriving models of the structures of proteins involve homology modeling. Homology modeling is applied to amino acid sequences that are evolutionarily related, i.e., they are homologous, such that their amino acid sequences can be aligned with some confidence. In one example of this method, the amino acid sequence of a protein whose structure has not been experimentally determined is aligned to the amino acid sequence of a protein whose structure is known using one of the standard sequence alignment algorithms (see, e.g., Altschul, et al. (1990), J. Mol. Biol., vol. 215:403-410; Needleman and Wunsch (1970), J. Mol. Biol., vol. 48:443-453; Pearson and Lipman (1988), Proc. Natl. Acad. Sci. USA, vol. 85:2444-2448). Homology modeling algorithms, for example, Homology (Molecular Simulations, Inc.), build the sequence of the protein whose structure is not known onto the structure of the known protein. The result is a computational model for the sequence whose structure has not been experimentally determined. Such a computational model of protein structure is termed a “homology model”. Preferred homology modeling methods are described in U.S. patent application Ser. No. 10/113,721, filed Mar. 30, 2002. See also Kolinski, et al. (2001), Proteins, vol. 44:133-149. In certain preferred embodiments of the invention, inexact protein structure models generated by homology modeling methods can be utilized to generate functional site profiles.

ii. Threading Algorithms

In an inverse folding approach to protein structure prediction, one “threads” a probe amino acid sequence through different template structures and attempts to find the most compatible structure for a given sequence. As one skilled in the art would recognize, any current threading algorithm, or those developed in the future could be used in conjunction with this invention.

In certain embodiments of threading algorithms, sequence-to-structure alignments are performed by a “local-global” version of the Smith-Waterman dynamic programming algorithm (Waterman, 1995). In such embodiments, alignments are ranked by one or more, preferably three, different scoring methods. In a three-method approach (Jaroszewski, et al., 1997), the first scoring method can be based on a sequence-sequence type of scoring. In this sequence-based method, the Gonnet mutation matrix can be used to optimize gap penalties, as described by Vogt and Argos (Vogt, et al., (1995)). The second method can use a sequence-structure scoring method based on the pseudo-energy from the probe sequence “mounted” in the structural environment in the template structure. The pseudo-energy term reflects the statistical propensity of successive amino acid pairs (from the probe sequence) to be found in particular secondary structures within the template structure. The third scoring method can concern structure-structure comparisons, whereby information from the known template structure(s) is(are) compared to the predicted secondary structure of the probe sequence. A particularly preferred secondary structure prediction scheme uses a nearest neighbor algorithm.

After computing scores for the sequence-to-structure alignments, the statistical significance of the each score is preferably determined by fitting the distribution of scores to an extreme value distribution, and the raw score is compared to the chance of obtaining the same score when comparing two unrelated sequences (Jaroszewski, et al., 1997).

Once the alignment of the probe sequence-to-template structure has been determined, a three-dimensional model can be built. A representative example of automated modeling tools include Modeller4 (Tripos Associates, St. Louis). Such tools preferably produce all non-hydrogen atom coordinate files for the three-dimensional model built from the sequence-to-structure alignment provided by the threading algorithm.

As will be appreciated, a final predicted structure is only as good as the sequence alignment produced by the threading algorithm, and local misalignments may occur in threading predictions and sequence alignments. This problem can be overcome in at least some cases by allowing for small errors in the alignments and by using not just the threading prediction with the highest score (i.e., the optimum alignment), but a number of top ranking, alternative threading-based structure predictions for the same sequence. When a threading algorithm is used in the practice of this invention, typically the sequence of a protein whose function is being evaluated is “threaded” through a large database (e.g., the PBD or a subset model protein structures therefrom) of proteins whose structures have been experimentally elucidated by, for example, XRD or NMR spectroscopy. A particularly preferred threading algorithm is the PROSPECTOR threading algorithm described in the U.S. patent application that claims the benefit of PCT/US01/30308. See also Skolnick & Kihara (2001),Proteins, vol. 42:319-331. A number of sequence-to-structure alignments are produced for each sequence. Each of these alignments, or preferably, 1, 2, 3, 4, or 5 of the highest scoring alignments, can be used to generate one or more protein models. These and other computational models of protein structures can be refined, as desired, using tools and methods available in the art.

iii. Ab Initio Structure Modeling

Another computational approach to protein structure elucidation involves ab initio prediction. Such procedures generally have two parts: 1) parameter derivation using information extracted from multiple sequence alignment; and 2) structure assembly (or “folding”) and refinement. As those in the art will appreciate, any conventional or later-developed ab initio protein structure prediction algorithm can be used in connection with this aspect of the invention.

In certain embodiments of the invention, the “MONSSTER” (Modeling Of New Structures from Secondary and Tertiary Restraints) ab initio folding algorithm is used to produce inexact models of protein structures. The MONSSTER algorithm uses a high coordination lattice-based α-carbon representation for the folding of proteins (Skolnick et al., 1997) and is modified to incorporate the expected accuracy and precision of the predicted tertiary structures (Ortiz et al., 1997). Parameters for ab initio folding, including predicted secondary and tertiary structure information, is extracted from multiple sequence alignment analysis. Other useful ab initio algorithms and refinement tools include those described in U.S. patent application Ser. No. 09/493,022, filed Jan. 27, 2000, as well as those described by Kihara, et al. (2001), Proc. Nat'l Acad. Sci., vol. 98(18):10125-30, and Feig, et al. (2000), Proteins, vol. 41:86-97; Ortiz, et al. (1999), CASP3 Proceedings, Proteins Suppl., vol. 3:177-185.

In certain preferred embodiments of the invention, inexact protein structure models generated by ab initio methods can be utilized to generate functional site profiles.

Functional Sites

In order to initially generate a functional site profile according to the invention, a functional site corresponding to, or that will be represented by, the functional site profile must be identified. Any suitable method for identifying functional sites that confer particular biochemical functions on proteins can be employed. In preferred embodiments, functional site identification involves identifying a small number of critical amino acid residues whose identities and structures are key to the chemical function of the particular functional site.

In certain embodiments, functional sites in proteins can be identified by reference to the scientific literature describing experimental results that indicate which amino acid residue(s) of the particular protein participate in, and preferably are critical for, the desired function. With information of this sort and a model (experimentally or computationally determined) of the structure of the protein (or a fragment thereof), a functional site profile according to the invention can be generated.

Particularly preferred methods for identifying protein functional sites employ functional site descriptors. Functional site descriptors define a spatial configuration for a protein functional site that corresponds to a biological function. Preferably, such descriptors are minimal descriptors, in that they contain only the minimum information needed to identify the desired functional site, but not other closely related structures that do not confer the corresponding biochemical activity on protein. Minimal descriptors are preferred because they will typically be implemented on a computer system for large scale analyzes. Such analyzes are typically computationally intensive. As such, it is desirable that the descriptors contain only that information necessary to distinguish proteins that exhibit the particular function from those that do not. Typically, such descriptors can be used to probe protein model structures, including those produced from data gathered using experimental structure solution techniques and those generated using any suitable computational method, to identify protein functional sites.

Preferred in the practice of the invention are structure-based functional site descriptors that describe (or represent) the relative three-dimensional orientation of two or more amino acid residues of a functional site can be employed for this purpose. Such functional site descriptors comprise, at a minimum, a spatial representation or configuration of at least two atoms, or groups of atoms, of different amino acid residues. By way of example, a functional site descriptor may be prepared using the interatomic distance, or preferably, a range of interatomic distances, between the α-carbon atoms of two amino acid residues known or suspected to be involved in the catalysis carried out by a particular enzyme. Alternatively, such a configuration can be represented in three dimensions using x, y, and z coordinates to identify the position, or range of positions, that a particular atom may have relative to other functionally important residues (or atoms of such residues).

The identity of functionally important amino acid residues, distances (or ranges of distances) between atoms or pseudoatoms, coordinate sets, or other parameter represent constraints with respect to the particular functional site descriptor. Preferably, a functional site descriptor includes one or more identity constraints, for example, the identity of a particular amino acid residue (or set of amino acid residues) located or predicted to be located at a particular position in a protein, in addition to a set of two or more geometric constraints. Other information can also be included, for example, information regarding bond angles (or bond angle ranges), secondary structure information, amino acid sequence, etc. For a more detailed description of how to make and use such functional site descriptors, see U.S. patent application Ser. Nos. 09/322,067 and 09/839,821.

As will be appreciated, functional site descriptors useful in generating functional site profiles can be developed for different functional site types. Particularly preferred functional sites are enzyme active sites, protein-protein interaction sites, sites for chemical modification, and ligand binding sites, e.g., metal ion binding sites, co-factor binding sites, antigen binding sites, substrate channels and tunnels, and substrate binding sites.

Pharmacophores

As described above, methods for designing as well as identifying compounds that specifically bind to a protein functional site defined by a functional site profile are among the many aspect of the invention. In certain embodiments of these aspects, such compounds may be defined by a predefined pharmacophore that accounts for the structure and chemistry of a protein functional site defined by a functional site profile.

Pharmacophores have proven to be highly valuable and useful in drug discovery and drug-lead optimization, as they define a distinct three-dimensional arrangement of chemical groups essential for biological activity at a target protein. Since a drug must interact with its target to be effective, and since the desired functional properties of the compound are derived from this interaction, each active compound must contain a distinct arrangement of chemical groups that enable this interaction to occur. The chemical groups, commonly termed descriptor centers, can be represented in suitable manner, for example, by (a) an atom or group of atoms; (b) pseudoatoms, for example a center of a ring, or the center of mass of a molecule; and (c) vectors, for example atomic pairs, electron lone pair directions, or the normal to a plane.

A pharmacophore can be constructed in a variety of ways. For example, the pharmacophore descriptor centers can be inferred from studying the X-ray or NMR structure of a protein-ligand complex, or by a shape-complementarity function analysis of the receptor binding site. This later approach involves the creation of an inverse pharmacophore based on the chemical groups presented on the surface of a protein functional site. When structural models of a protein-ligand complex derived by experimental methods are not available for the target protein, an inverse pharmacophore for the functional site can be developed and used to guide the creation of one or more complemetary pharmacophores. Such complementary pharmacophore can then be used to screen one or more three-dimensional virtual compound libraries. As will be appreciated, pharmacophores and inverse pharmacophores, and the methods described herein for making and using them, represent additional aspects and embodiments of the invention.

Typically, the pharmacophore is used to screen a virtual library of compound structures defined by three-dimensional coordinates in order to identify one or more compounds that match the pharmacophore. Often, the compounds represented in a virtual library are comprised of one or more scaffold(s) that display substituents that alone or together with scaffold atoms fit the chemical and structural properties or constraints defined by the pharmacophore. See, e.g., U.S. Pat. No. 6,343,257.

A three-dimensional virtual compound library can be made or derived from any suitable source, for example, a commercially, publicly available source, as well as from non-public sources (for example, a pharmaceutical company's library of proprietary and/or non-proprietary compounds). The compounds in the library may be existing compounds and/or virtual compounds, i.e., those existing solely in a computer. The library may also be a virtual combinatorial library (VCL) the members of which are constructed from a virtual library of scaffolds and a virtual library of substituents that can be placed at each of a set of predefined attachment positions on each scaffold. See U.S. Pat. No. 6,343,257. In some cases, a compound library may not provide three-dimensional representations of its members, in which event three-dimensional representations of the compounds in the library will be generated using any suitable technique.

The screening process proceeds by filtering out compounds whose structures and chemistries are incompatible with the pharmacophore. At the end of the process, those compounds that remain will have structures that match the structural and chemical criteria of the desired pharmacophore. As will be appreciated, pharmacophores and inverse pharmacophores represent additional

Compounds

While not being bound by any particular theory, it is believed that the compounds identified in accordance with the methods of the invention bind covalently or non-covalently to a protein by specifically interacting with the chemistry and structure present in a functional site in the protein that corresponds to the functional site profile used to design or identify the compound.

The compounds of the invention may be synthesized using conventional techniques. Advantageously, these compounds are conveniently synthesized from readily available starting materials. The compounds of this invention may contain one or more asymmetric carbon atoms, and thus may occur as racemates and racemic mixtures, single enantiomers, diastereomeric mixtures, and individual diastereomers. All such isomeric forms of these compounds are expressly included in the present invention. Each stereogenic carbon may be of the R or S configuration. Combinations of substituents and variables envisioned by this invention are only those that result in the formation of stable compounds, i e., compounds which possess stability sufficient to allow manufacture and which maintain their integrity for a sufficient period of time to be useful for the intended purpose (e.g., for therapeutic or prophylactic administration to a mammal, for use in medicinal chemistry programs to generate derivatives and analogs, etc.). Typically, such compounds are stable at a temperature of 40° C. or less, in the absence of moisture or other chemically reactive conditions, for at least a week.

As used herein, the compounds of this invention include useful derivatives and analogs, as well as useful salts, esters, and salts of esters. Also included are activatable forms of the compounds, e.g., prodrugs, which can be activated when placed under suitable conditions. Also envisioned are derivatives designed to enhance biological properties such as oral absorption, clearance, metabolism, or compartmental distribution. Typically, such derivatives are made by appending appropriate functionalities to a compound to enhance selective biological properties. Such modifications are known in the art and include those which increase biological penetration into a given biological compartment (e.g., blood, lymphatic system, central nervous system), increase oral availability, increase solubility to allow administration by injection, alter metabolism, and alter rate of excretion.

Useful salts of the compounds of this invention include those derived from useful inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic acid, may be employed in the preparation of salts useful as intermediates in obtaining the compounds of the invention and their useful acid addition salts.

Salts derived from appropriate bases include alkali metal (e.g., sodium and potassium), alkaline earth metal (e.g., magnesium), and ammonium salts. This invention also envisions the quaternization of any basic nitrogen-containing groups of the compounds disclosed herein. Water or oil-soluble or dispersible products may be obtained by such quaternization. In some cases, the pH of the formulation may be adjusted with acceptable acids, bases, or buffers to enhance the stability of the formulated compound or its delivery form.

The compounds of the invention (including useful salts thereof) can be included in compositions that also contain one or more other components, e.g., any acceptable carrier, adjuvant, or vehicle. The terms “acceptable carrier” and “acceptable adjuvant” refer to carriers and adjuvants, respectively, suitable for introduction into an animal, together with a compound of the invention, and which does not destroy the modulating activity of the compound and is nontoxic when provided in an amount sufficient to produce a desired effect.

Acceptable carriers, adjuvants, and vehicles that may be used in the compositions of this invention include ion exchangers, alumina, aluminum stearate, lecithin, self-emulsifying drug delivery systems (SEDDS) such as d-α.-tocopherol, polyethyleneglycol 1000 succinate, surfactrants such as Tweens or other similar polymeric matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle- ne-block polymers, polyethylene glycol, and wool fat. Cyclodextrins, including chemically modified derivatives such as hydroxyalkylcyclodextrins, or other solubilized derivatives may also be used.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. A sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringers solution, and isotonic sodium chloride solution. In addition, sterile, fixed oils are conventionally employed as a solvent or suspending medium. For this purpose, any bland fixed oil may be employed, including synthetic mono- or diglycerides. Fatty acids, such as oleic acid and its glyceride derivatives, are useful in the preparation of injectables, as are natural oils, such as olive oil or castor oil, especially in their polyoxyethylated versions. These oil solutions or suspensions may also contain a long-chain alcohol diluent or dispersant or a similar alcohol, or carboxymethyl cellulose or similar dispersing agents that are commonly used in the formulation of dosage forms such as emulsions and/or suspensions. Other commonly used surfactants such as Tweens or Spans and/or other similar emulsifying agents or bioavailability enhancers which are commonly used in the manufacture of solid, liquid, or other dosage forms may also be used for the purposes of formulation.

Typically, the amount of a compound to be delivered is between about 0.01 and about 100 mg/kg body weight per day, preferably between about 0.5 and about 75 mg/kg body weight per day. A suitable administration interval is used. As those in the art appreciate, the amount of active ingredient that may be combined with carrier materials to produce a single dosage form will vary depending upon the intended application and particular mode of administration. A typical preparation will contain from about 5% to about 95% active compound (w/w). Preferably, such preparations contain from about 20% to about 80% active compound.

As the skilled artisan will appreciate, lower or higher doses than those recited above may be required. Specific dosage and administration regimens will depend upon a variety of factors, including the activity of the specific compound employed, the age, body weight, general health status, sex, diet, time of administration, rate of excretion, drug combination, the severity and course of the condition, the patient's disposition to the infection and the judgment of the treating physician.

The compounds set forth herein may also be used as laboratory reagents, e.g., as compounds for co-crystallization with the protein with which the compound specifically interacts. Alternatively, they may represent “hits” or “leads” identified in an initial stage of a drug discovery process. Analogs of such compounds may be generated by a program of medicinal chemistry to alter one or more attributes of the compound. Iterative steps of drug design, synthesis, and modification can be employed until a compound having the desired properties is generated.

Computer-Implemented Embodiments of the Invention

The various techniques, methods, aspects, and embodiments of the invention can be implemented in part or in whole using computer-based systems and methods. Additionally, computer-based systems and methods can be used to augment or enhance the functionality described above, increase the speed at which the functions can be performed, and provide additional features and aspects as a part of or in addition to those of the present invention described elsewhere in this document. Representative computer-based systems, methods, and implementations in accordance with the above-described technology are now presented, although as will be appreciated, any suitable system may be employed to implement the instant invention. Accordingly, this description is not intended to, and should not be construed as, implying a particular physical, logical, or structural architecture for implementing computer-based systems to carry out the invention. In fact, it will be apparent to one of ordinary skill in the art after reading this detailed description how to implement the various features and aspects of the invention using any suitable alternative processor architectures and configurations, including alternative combinations and configurations of computer software and hardware.

The various embodiments, aspects, and features of the invention may be implemented using hardware, software, or a combination thereof, and may be implemented using a computing system having one or more processors. The system can include one or more memories to allow computer programs or other instructions or data to be loaded into the computer system. Preferred memories include random access memory (RAM). One or more secondary memories can also be included. Secondary memory includes hard disk drives and removable storage devices such as floppy disk drives, magnetic tape drives, optical disk drives, etc. Typically, a removable storage drive reads from and/or writes to a removable storage medium. Removable storage media include floppy disks, magnetic tapes, optical disks, cartridges, removable memory chips, etc. that can be from read and written to. As will be appreciated, the removable storage media includes a computer usable storage medium having stored therein computer software and/or data.

A computer system can also include communications interfaces to allow software and data to be transferred between computer system and external devices. Examples of communications interfaces include modems, network interfaces (such as, for example, an Ethernet card), communications ports, PCMCIA slots and cards, etc. Software and data transferred via a communications interface typically are in the form of signals that can be electronic, electromagnetic, optical, or other signals capable of being received by the communications interface. Signals are typically provided to communications interfaces via one or more channels. Channels carry signals and can be implemented using a wireless medium, wire or cable, fiber optics, or other communications medium. Some examples of a channel can include a phone line, a cellular phone link, an RF link, a network interface, and other communications channels.

In this document, the terms “computer program medium” and “computer usable medium” are used to generally refer to media such as removable storage devices (e.g., a disk capable of installation in a disk drive) and signals on channel 528. These computer program products and the like allow software, program instructions, and data to be provided to the computer system.

Computer programs (also called computer control logic) typically are stored in a main memory and/or secondary memory. They may be provided by way of removable storage media or embedded in hardware (e.g., in an application specific integrated circuit (ASIC)) or other hardware component. Computer programs can also be received via a communications interface. Computer programs, when executed, enable the computer system to perform the features of the present invention as discussed herein by manipulating and processing data in accordance with the encoded computer program logic. Accordingly, computer programs represent controllers of the computer system.

The examples below are provided to illustrate the subject invention, and in no way limit the its scope.

### EXAMPLES

The following examples are provided to illustrate the practice of certain preferred embodiments of the instant invention.

### Example 1

### Structure-Based Functional Site Profiles and Family Sub-Classification

1. Introduction

Large-scale function assignment for proteomic studies such as expression monitoring traditionally has relied on automatically transferring annotation from the most similar or related protein sequence to uncharacterized proteins of interest. However, as discussed above, transfer of annotation based on whole or overall sequence comparisons or the use of sequence motifs (e.g., Blocks, Pfam, and Prints) is inadequate. The instant invention circumvents the limitations of conventional overall sequence comparisons and sequence motif based methods.

This example focuses on certain aspects and preferred embodiments of the invention, namely the generation of structure-based functional site profiles useful in whole-genome analyzes and protein sub-family classification. In this example, structure-based functional annotation methods using functional site profiles are coupled with computational structural analyses performed using certain functional site descriptors commonly referred to as “Fuzzy Functional Forms” or “FFFs”. See Fetrow & Skolnick (1998), J Mol Biol., vol. 281, 949-968; U.S. patent application Ser. No. 09/322,067, filed May 27, 1999. Briefly, the described in this example began by detecting the fold of a query protein sequence using a threading algorithm capable of whole-genome throughput and analysis. Skolnick & Kihara (2001), Proteins, vol. 42(3), 319-331; PCT/US01/30308. Next, the resulting structure models were scanned for the presence and correct geometries of key amino acids responsible for biochemical activity, substrate or cofactor binding, or catalytically important metal binding using a library of FFFs. Fetrow, et al. (1998), J Mol Biol., vol. 282(4), 703-711; Fetrow, et al. (1999), FASEB J., vol. 13(13), 1866-1874; Fetrow & Skolnick (1998). Development of an FFF does not depend on multiple sequence alignments or sequence pattern identification. Rather, each FFF is hand-curated and validated using databases of protein structures and experimental literature. Conserved residues important for fold rather than function are specifically excluded from the functional site definition. As a result, FFF-based functional site profiling has the advantage of automatically identifying functionally significant residues clustered in three-dimensional space within the protein structure. In this way, a precise determination of protein function, based on functional sites only, was made and the physiochemical properties of the functional sites were characterized. In addition, the FFF approach allows unambiguous assignments of multiple functional sites to be made in a single protein structure.

As described in this example, the methods of the invention were applied to extend the functional site-centered method for functional annotation to functional family classification. Successful sub-type classification speeds the design of experimental assays for function confirmation and guides searches for appropriate small molecule modulators of protein function (e.g., inhibitors). As will be appreciated, in this example, for each protein family, the collective ensemble of active site residues, based on the FFF-defined active site templates, forms a functional site profile. The functional site profile encodes key similarities and distinguishing features between functional sites, enabling sub-family assignment and providing information relevant to structure-based drug design of selective ligands.

This work demonstrates the significance of functional site profiles based on FFFs. Validation studies in four areas highlight the utility of these profiles in addressing challenges of genomic analysis and function assignment. First, it is shown that FFF-based functional site profiles distinguish true positive functional sites from decoy sites that exhibit similar conservation of features and geometries, but do not perform the same function. Second, it is shown that FFFs are unique by performing an all-against-all comparison of 193 distinct functional site profiles. Importantly, it is also shown that the profiles can accurately assess threaded protein structure models, and that the profiles enable automatic sub-family classification. Finally, the methods are shown to be effective at recognizing the likely sub-family membership for large protein families, even when the average conservation of the functional site residues across the entire family is low. The methods were applied to a diverse family of protein kinases, and in most cases, they identified the sub-class indicated by literature review. Additionally, the study also identified previously uncharacterized kinases for which functional site analysis suggests a different sub-family than classification based on overall sequence similarity.

2. Results

A. Structure-Based Functional Site Descriptors Identify Protein Active Sites

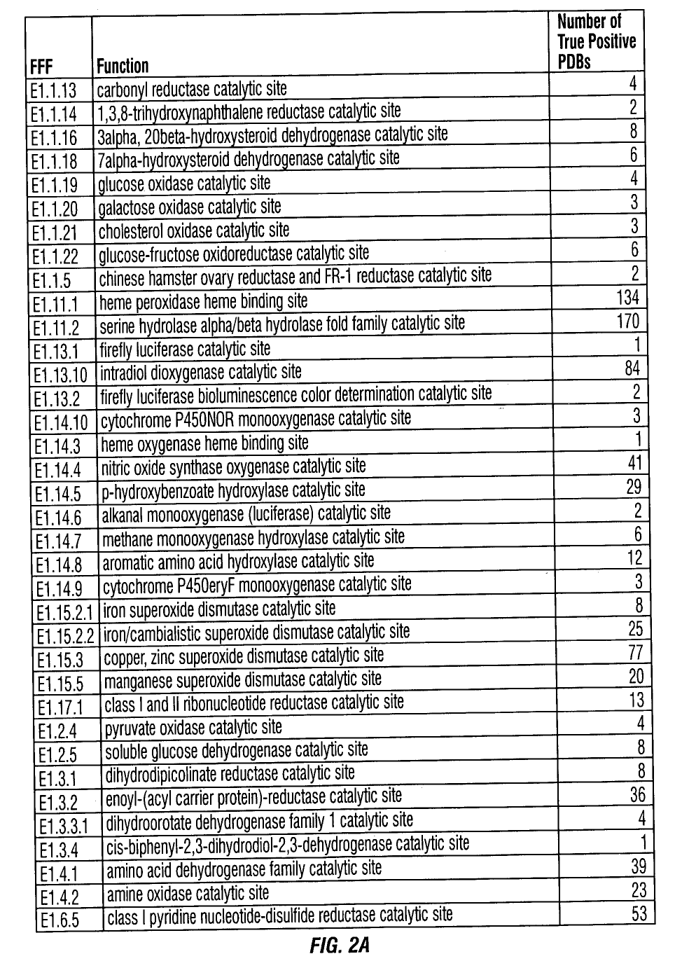

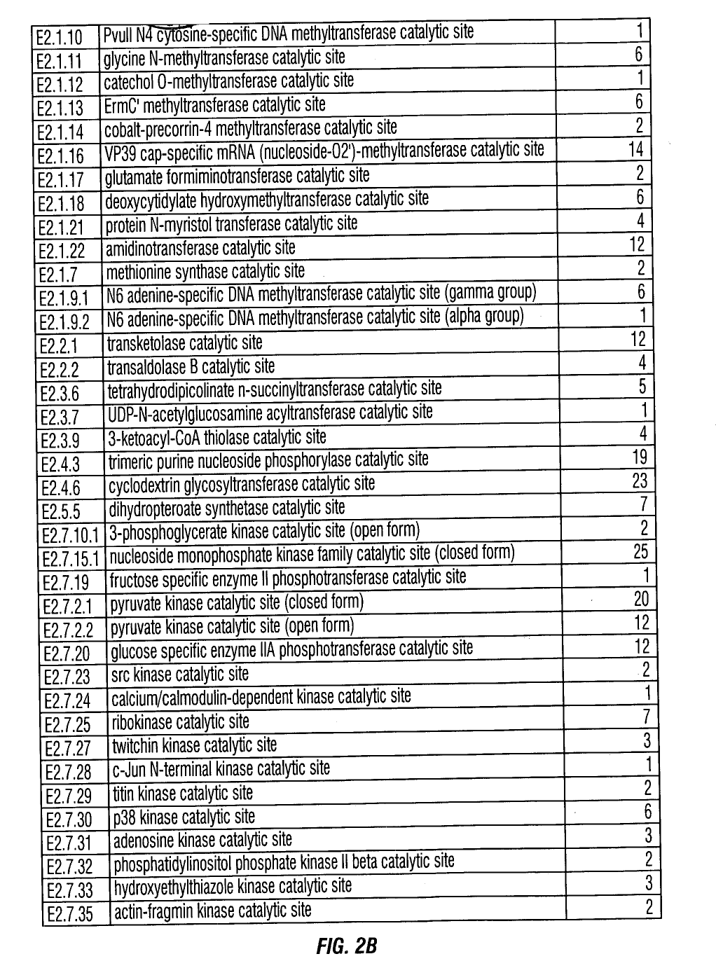

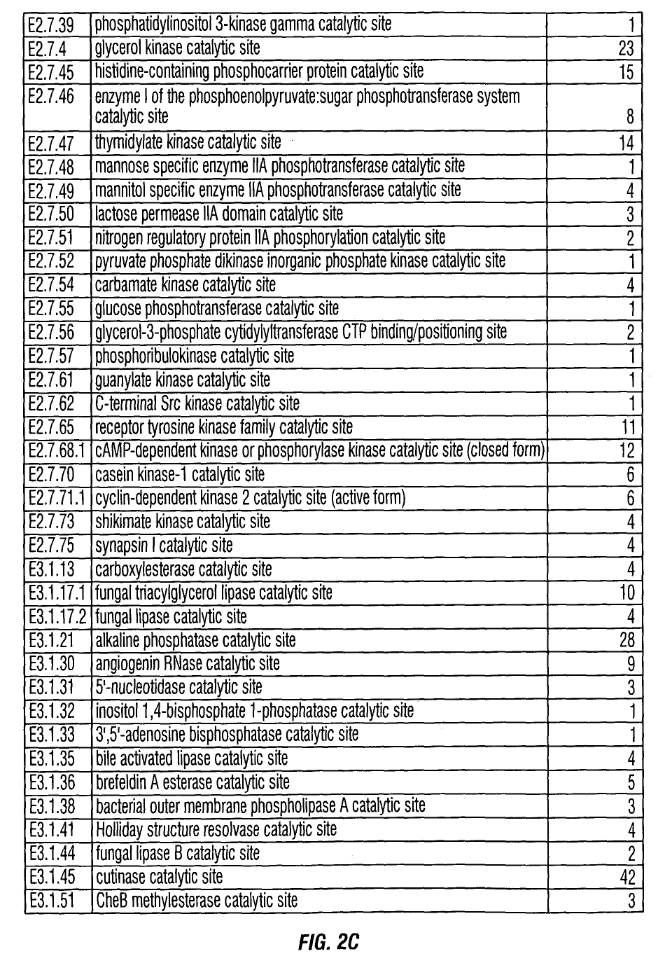

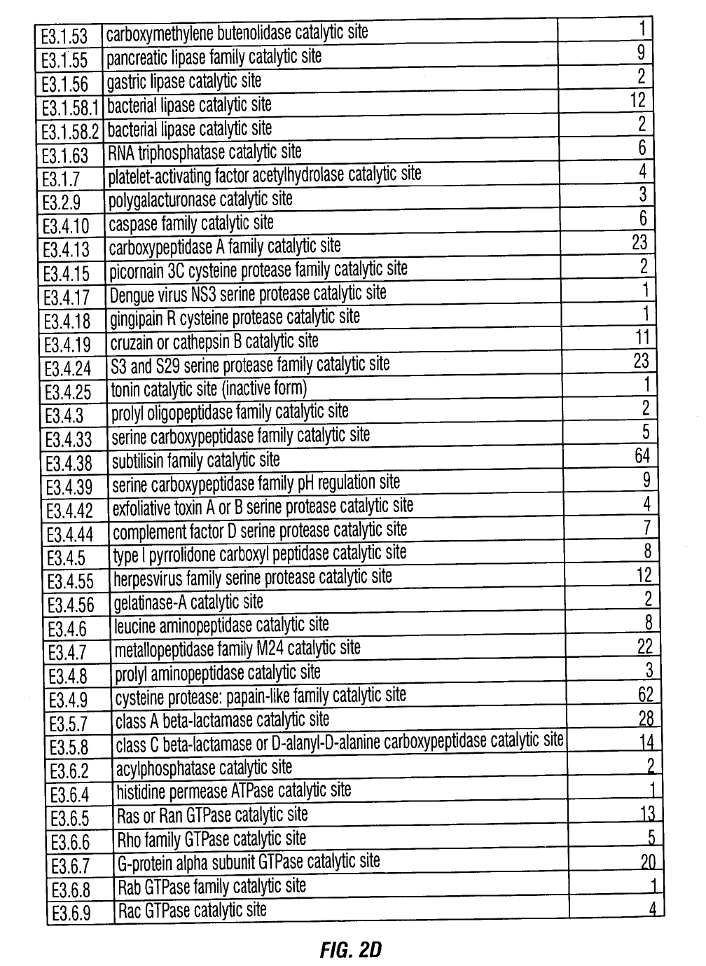

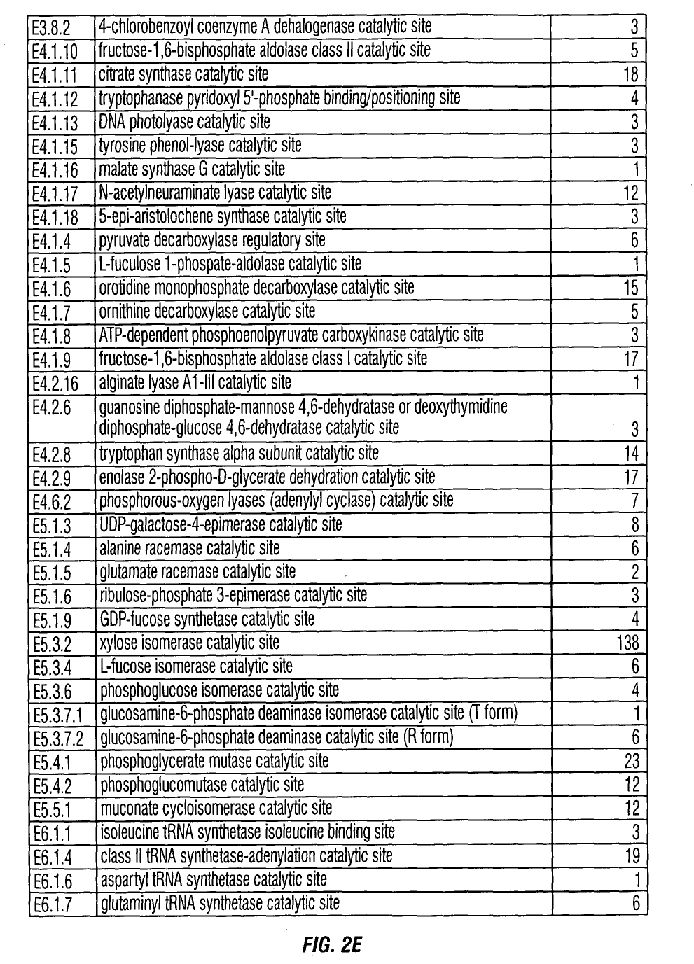

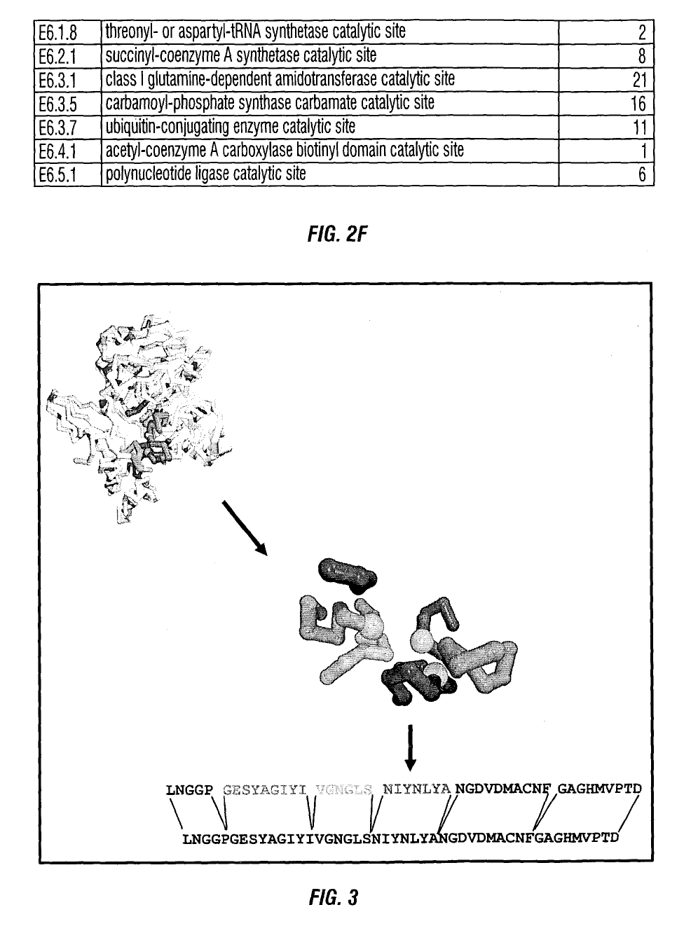

Here, functional site recognition began with FFF construction for active sites. FFFs were designed from the three-dimensional structural arrangements of functionally important residues that are essential to the biochemical mechanism underlying the enzymatic function. The 193 FFFs used here describe active sites that carry out a wide range of enzymatic activities (FIG. 2) and represent functions across all six Enzyme Commission (EC) classes (IUBMB, 1999).

Each FFF was validated by automatically applying it to structure coordinates available in the PDB. Optimization of the structural discrimination metric (Fetrow, et al., 1998) ensured that all true positive protein structures were recognized, while false positives were excluded. Most (72%) FFF definitions used in this study included more than two true positive protein structures from the PDB.

Decoy protein structures in the PDB were identified by loosening the geometric constraints of the validated FFFs. These decoy structures contained somewhat similar spatial arrangements of key residues identified by the FFFs, but differed significantly in overall configuration and residue conservation at the active site. In addition, no available experimental evidence existed to suggest that the proteins corresponding to the decoy structures indeed carry out the biochemical function described by the validated FFF. The closest, or nearest, decoy structures in the PDB were identified for each of the 193 FFFs used in this study.

B. Active Site Profile Construction

The true positive protein structures recognized by each FFF were used to construct function site profiles for the corresponding active sites. The key functional residues identified by the FFF in each structure were used to extract active site sequence fragments from the protein structure (FIG. 3). Sequence fragments were then joined as one string according to their primary sequence ordering, forming the function site (here, active site) signature for each function. The active site signatures captured residues within a 10 Å radius from the key functional residues identified by each FFF. A default 10 Å radius was chosen so that a sufficient number of residues in the spatial environment of the particular active site was included to make the signature unique and characteristic. In addition, the size of the sphere was generous enough to allow analysis of computationally generated structure models.

The active site signatures from all protein structures recognized by a given FFF were aligned to yield a multiple sequence alignment, constituting the active site profile. The serine carboxypeptidase family active site profile is shown in FIG. 3. The active site profiles revealed relative conservation of residues within a closely related protein family. As in any multiple sequence alignment, some conserved residues play key functional or mechanistic roles in protein activity while others may be important for structure or folding. However, the active site profiles are enriched with residues important for enzymatic activity since they are based on key functional residues defined by the FFFs. Differences in active site geometry resulted in gaps in the active site signature, reflecting the absence of residue positions within the default 10 Å radius. Differences in amino acid identity and active site configuration likely impact relative protein activity and substrate specificity of the proteins recognized by a given FFF. For instance, while the serine carboxypeptidase FFF recognized a set of proteins that carry out the same active site chemistry, the substrate specificity differs among the set. Wheat carboxypeptidase II stabilizes the positive charge of Lys and Arg residues on the substrate (Liao, et al. (1992), Biochemistry, vol. 31(40), 9796-812), while the active site of carboxypeptidase Y is nonpolar, and cannot accommodate charged substrates (Endrizzi, et al. (1994), Biochemistry, vol. 33(37), 11106-20). Nonetheless, the serine carboxypeptidase FFF used accommodates these divergent family members.

C. Active Site Profile Scoring

Active site profile scores were calculated to evaluate residue and structure conservation at each position within a FFF-based family of functional sites. The active site profiles contain amino acid sequences of the true positive structures recognized by the FFF, as in the serine carboxypeptidase family of structures shown in FIG. 3A. Active site profile scores were calculated as described below (see Materials and Methods) for 193 functional families of structures, defined by the FFF set used in this study. Briefly, the variation of residue types for each active site profile position was evaluated using the following four conditions: identity; strongly conserved; weakly conserved; and gapped positions. Scores for all functional site residues were summed and normalized to generate a profile score where a score of 1.0 indicates 100% identity among a group of active sites. A profile score of 0.0 or less corresponds to little or no similarity among an FFF-based functional family.

For 23 of the 193 FFF-based functional families examined in this study, the active site profile scores were below 0.30 (approximately 30% identity), indicating high variation of residues within the spatial environment defined the 10 Å radius among the proteins in these families. For some of these functional families, such as the serine hydrolases (Ollis, et al. (1992), Protein Eng 5(3), 197-211), multiple folds covering a broad range of different, but biochemically-related, functions were included in the active site profiles; thus, low profile scores for these families were not unexpected.

Active site profiles can be used to evaluate active site structures in either experimentally determined structures (Fetrow & Skolnick, 1998) or computationally modeled structures (Di Gennaro, et al. (2001), J Struct Biol., vol. 134(2-3), 232-245). Using FFFs to identify functional sites, active site sequence signatures can be extracted and compared to FFF-based active site profile alignments. A family conservation score can be calculated for the functional site profile aligned with the query sequence signature, and by comparing the family score to the profile score, a quantitative measure of the similarity of the query sequence to the active site profile can be obtained. A pairwise score can be calculated by retaining the maximum score for the alignment between the query sequence signature and each true positive signature included in the active site profile. The maximum pairwise score identifies the structurally characterized family member most closely related to the query sequence. In this way, the most closely related active site structure in a functional family could be identified, enabling a more detailed sub-classification of query proteins, depending on the number of functional family protein structures available in the PDB. As expected, in most cases query sequences had higher pairwise alignment scores compared to the calculated family score. Nonetheless, low family and pairwise scores merely reflected a low similarity of the query structure to experimentally determined, or known, structural space.

D. Active Site Profile Scoring Allows Discrimination of True and False Active Site Structures

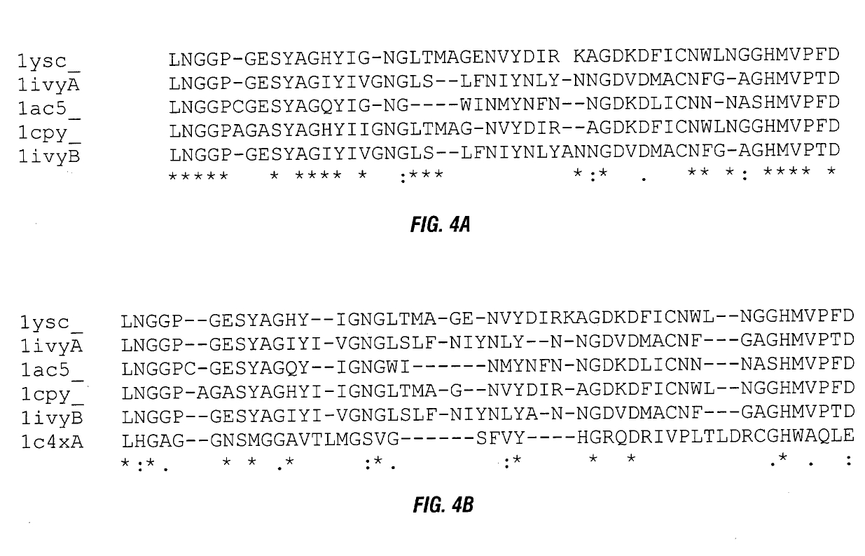

FFF-based active site profiles can also be used to discriminate between true functional site structures and decoy sites in protein structures. Closest, or most similar, decoy active site structures in the PDB were identified for each of the 193 FFF-based functional families used in this study. For each functional family, the sequence of the closest decoy active site available in the PDB was aligned against the active site profile. The similarity of the alignment between the false, or decoy, active site and the active site profile was evaluated by calculating a family conservation score, as discussed above. As an example, the closest decoy structure identified for the serine carboxypeptidase functional family was 2-hydroxy-6-oxo-6-phenylhexa-2,4-dienoate hydrolase (1c4x, FIG. 4B). The query family score for 1c4x was 0.14, a much lower score than the active site profile score 0.41 for the serine carboxypeptidase functional family. The lower score is consistent with the fact that 1c4x does not have peptidase function, although it is a member of the serine hydrolase superfamily.

Decoy active sites had query family scores (FIG. 5A) ranging from a low of 0.19 to a maximum of 0.27. Of these, only eight decoy signatures aligned with a score greater than 0.2. For each of the eight highest scoring decoy structures, the scoring difference between the family score and the active site profile score exceeded 0.50. For more than 90% of the FFF-based functional families, the difference between active site profile scores and decoy family scores exceeded 0.25, suggesting that this score could be a confidence cutoff for the recognition of true active sites.

FIG. 5B depicts cases in which the closest decoy structure identified actually exhibited a biochemical function related to the FFF-based functional family members. For example, the nearest decoy for the chinese hamster ovary/FR-1 reductase catalytic site FFF is 1ah3, aldose reductase, clearly a member of the same superfamily of enzymes. The decoy family score in this case was 0.83 and the profile score was 0.94, indicating a high degree of active site similarity between the decoy and the subfamily members. The difference between the scores for a decoy active site signature and the active site profile was usually much higher (on average 0.51). The query family scores for the decoy signatures ranged from 0.23 to 0.83, with a mean score of 0.36. These functionally similar decoy structures scored in the range of true active site profile scores calculated for some FFF-defined functional families (FIG. 5A).

E. All-Against-All Comparison of FFF-Based Active Site Profiles Demonstrates Uniqueness

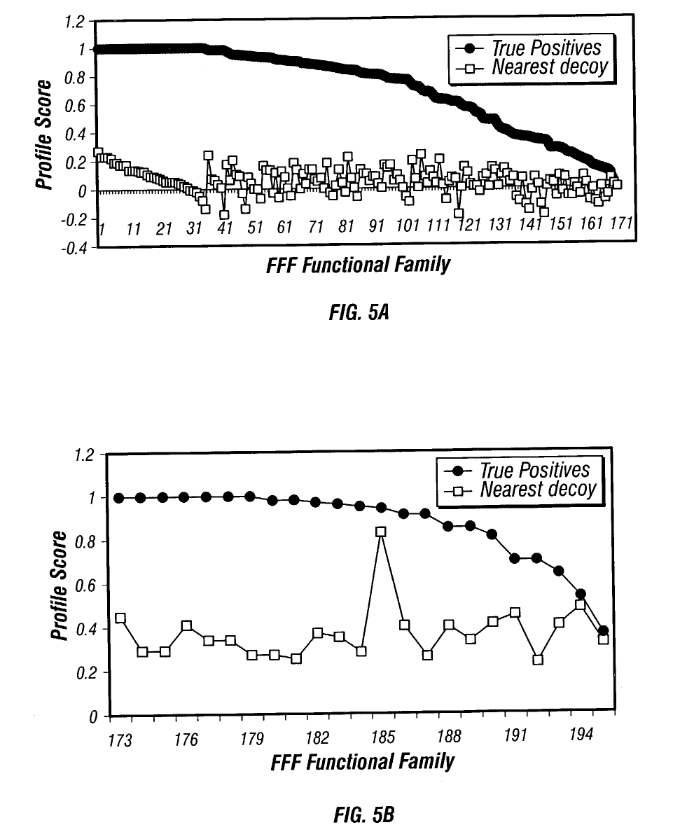

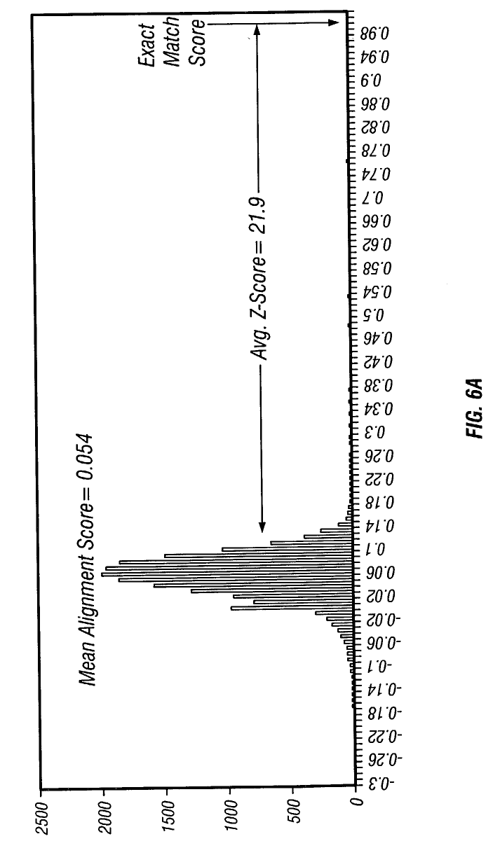

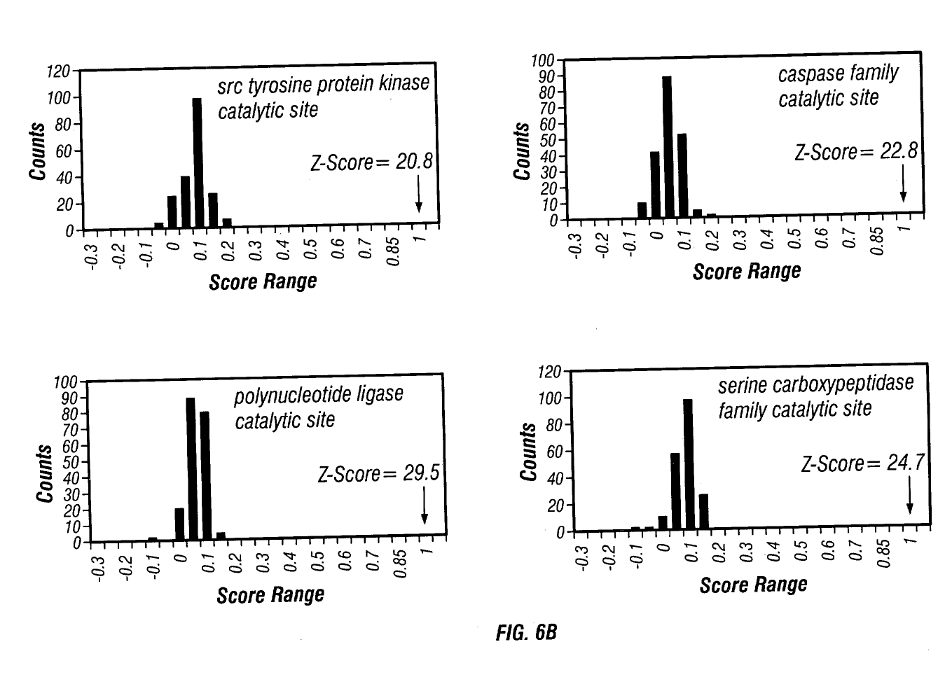

To demonstrate that the FFF-based functional site profiles were unique, an all-against-all comparison of the active site profiles was performed. For each of the 193 FFF-based functional family active site profiles, one representative protein structure and its associated active site signature were selected. The active site signature sequences were aligned to all other active site profiles, generating 18,528 alignments. Each query signature alignment was scored against all other active site profiles using the pairwise scoring method, as described above. The complete ensemble of alternative alignment scores were tabulated for each FFF-based functional site profile and used to calculate both a mean value and standard deviation for the distribution. These parameters then were used to translate the scores obtained for alignment of the corresponding active site to itself (self-alignment score=1.0, or 100% identity) to a Z score, expressed as the number of standard deviation units the self-recognition score was from the mean of the ensemble of scores (FIG. 6). For this test, a Z score of 5.0 or greater was designated as the threshold for significant discrimination of the active site signature with respect to all others.

The all-against-all comparison demonstrated that the average active site self-recognition score was resolved from the nearest mismatched pairwise score by nearly 22 SD units, indicating that the functional site profiles are highly unique. The distribution for the alternative alignments is visually normal (FIG. 6), ensuring that the translation of scores to a Z score was reasonable. The mean score for an alignment to any other distinct active site was only 0.054, whereas a self-recogniation score (100% identity) is 1.0. Thus, functional sites defined according to the FFF-based method were not merely random clusters of residues proximal to key active site amino acids; rather, the functional site profiles uniquely described structural and chemical environments and are appropriate for evaluating FFF-recognized functional sites on protein structures and models. More importantly, the distribution of alternative alignment pairwise scores established a level at which predicted site scores should be very highly significant—specifically, a pairwise score value of about 0.30 (or greater than 5 SD) would indicate similarity to a known protein active site structure. Pairwise or family scores above 0.5 would lend high confidence to a FFF-based functional site assignment in a novel protein structure or computationally determined model.

F. Benchmarking Active Site Profile Scoring Using Genomic Test Set

Active site profiles can be used to evaluate functional site assignments based on low-resolution models generated automatically, for example, by the PROSPECTOR threading algorithm, in combination with functional site descriptors such as FFFs. Here, a test set of 716 human genome sequences was identified for which confidence in the functional annotation was high, based on the consensus of a variety of annotation methods, but for which experimental structures were not available. Four sequence-based methods, Blocks, BLAST PRINTS, and Pfam, in addition to the structure-based PROSPECTOR/FFF method, were used to make biochemical function assignments. High-confidence annotation was based on all methods making the same functional assignment.

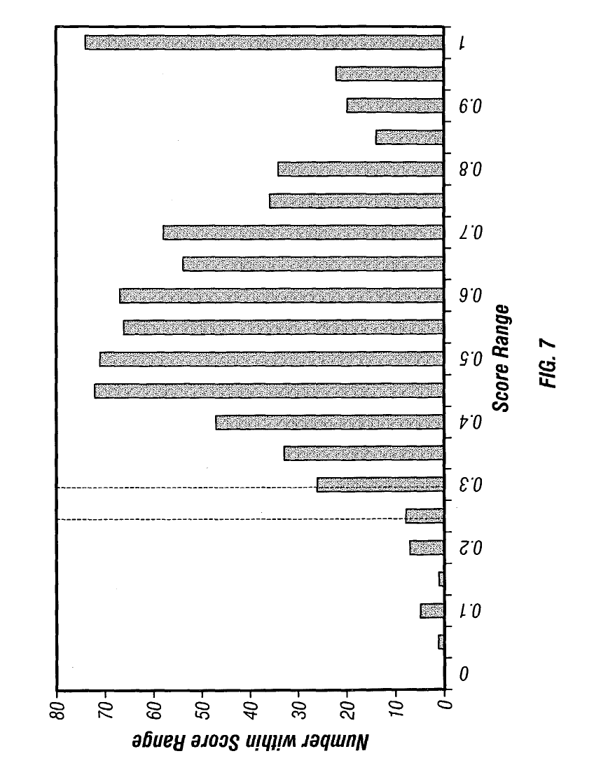

As indicated in FIG. 7, sequences with consensus functional annotation produced highly significant FFF-based active site pairwise scores for the ensemble of computationally discovered active sites. The mean pairwise score for these functional site models was 0.60, indicating a mean Z score of 7.03, when compared to the distribution of functional site mismatches established above. Sequence identity of approximately 25% is often taken as the threshold for recognition of meaningful similarity. This threshold translates to a family score of 0.25. While 694 of the consensus annotated sequences scored above this recognition threshold of 0.25, 20 of the modeled active sites scored below this measure. These low scoring functional assignments represented a small population (less than 3%) of the total set, and may have included lower quality models, or perhaps, false positives. Alternatively, these functional assignments may indeed identify proteins distantly related to the known protein structures in the PDB.

G. Assessment of Sub-Family Functional Assignment for Protein Kinases

To highlight the advantage of performing pairwise scoring to subclassify functional sites, active site profiling was applied to a set of protein kinase sequences. Protein kinases are members of a large functional family that have a conserved catalytic site that carries out phosphoryl transfer reactions (Hanks & Hunter (1995), Faseb J., vol. 9(8), 576-96). They are distinguished from each other by features affecting their biochemical activity, such as regulatory or substrate specificity sites. Most of these auxiliary functional sites are proximal to the catalytic site and may be reflected in the active site profiles. An FFF for the protein kinase catalytic site was constructed and validated against 82 protein kinase structures in the PDB. When applied to threaded models from a whole human genome analysis, the kinase FFF identified 589 sequences.

The alignment of the 589 putative kinase functional sites with the kinase family active site profile yielded low family scores due to the diversity observed in the active site. The average family score for the predicted kinase active site signatures was 0.07. However, the pairwise scores were much higher, as was the average pairwise score of 0.59. Transferring the classification of the true positive active site structure with the highest sub-family score yielded a sub-classification assignment. Sub-classifications determined in this manner were consistent with the accepted grouping of each kinase (Hanks & Hunter, 1995), except in those cases in which the group is not represented well in the structural database or the kinase structure is currently uncharacterized or unclassified.

This active site-based sub-classification method was very accurate in cases in which the accepted kinase sub-classifications were curated on the basis of active site features rather than global sequence similarity for the entire kinase domain. For example, the active site-based sub-classification assignments correctly classified the serine/threonine kinase AMP-Activated Protein Kinase (AMPK), a kinase involved in the regulation of fatty acid and cholesterol metabolism, as a member of the Calcium/Calmodulin (CAMK) group. This is a known example in which an accurate sub-classification can be determined readily using information in the literature, but use of motif-based tools would likely result in a misclassification. Both Blocks and PRINTS classify this kinase as a tyrosine kinase and yield no further information. A Blast analysis agrees with the active site-based sub-classification of AMPK in the CAMK group of Ser/Thr kinases, finding strong homology to the known CAMK group member SNF1 (Morrison, et al. (2000), J Cell Biol., vol. 150(2), F57-62) and related kinases in yeast, rice, and cucumber (Takano, et al. (1998), Mol Gen Genet., vol. 260(4), 388-394).

A significant percentage of kinases in the human genome do not show a clear relationship to a characterized protein kinase, and finding the best structural representatives for these kinases is not possible using purely sequence-based techniques. The FFF-based sub-classification method yields valuable information about the structure of the active site of many of these poorly characterized kinases. For instance, a Blast analysis was unable to classify IKK-related kinase epsilon (IKKε), a recently discovered Ser/Thr kinase thought to be involved in immune and inflammation responses (Peters & Maniatis (2001), Biochim. Biophys. Acta., vol. 2(62), M57-62). The top twenty Blast hits were all to other unclassified IKK kinases. Other Blast hits with E-values less than 10⁻²⁰ fell into the CMGC, AGC, and other Ser/Thr kinase groups. Blocks and PRINTS both misclassified this kinase as a tyrosine kinase. In marked contrast, the instant active site-based sub-classification method correctly assigned IKKε as a Ser/Thr kinase, and was further able to sub-classify it into the CAMK group.

3. Discussion

The above results demonstrate that a library of structure-based functional site descriptors can be used in conjunction with the methods of the invention, for example, to group protein families based on active site geometry and physicochemical properties, rather than overall sequence or structure. As described, FFFs for 193 different enzyme families were created and automatically applied to both protein structures and computationally derived models.

However, since FFFs encapsulate only a small amount of physical and chemical structural elements for a specific biochemical function, use of the instant invention allows such methodology to extended to include the some or all of the active site environment, which is represented as a functional site profile. These functional site profiles are unique and characteristic of the corresponding functional site, e.g., an active site; they are not simply random collections of residues proximal in space to the key biochemical features. In addition, the experiments described above establish that functional site profiles can be derived from protein models generated by methods for computationally predicting protein structures, such as by threading algorithms, comparative or homology modeling techniques, and ab initio methods. Here, the models generated by threading methods were approximations of the structure of the query sequence based on an alignment to a structure template. By extracting a large radius around key active site residues, sufficient surrounding structure was sampled to encompass the functional site, even in cases where the threading alignment may have contained local errors, demonstrating the relative insensitivity of the method to structural differences between predicted and experimentally determined active sites. Accordingly, this method allows active site profiling even for uncharacterized proteins identified by sequencing projects and expression monitoring studies.

The ability to apply functional site descriptors and active site profiling to threading-based models enables coverage similar to that of sequence-based methods, while retaining the structural insights available from methods that can only be applied to experimental structures.

It is also important to note that pre-defined confidence cutoffs, such as those described above, can be used in large-scale applications to help identify functional sites in proteomic sequences that are similar to a structurally characterized functional family member. In the above study, it was demonstrated that signatures with a high pairwise active site score (greater than about 0.25) for a particular family had a high likelihood of belonging to that functional family. However, many functional families are not fully characterized in the current structural databases, and, therefore, newly discovered functional family members may well be found to have low-scoring active site signatures. Moreover, there has been experimental confirmation of the function of proteins with pairwise active site scores lower than 0.25. Consequently, the scores described above are not absolute, and lower thresholds may be used successfully. Above, a cutoff of 0.25 used to prioritize proteins for further study (e.g., for cloning, expression, biochemical and biophysical analysis, experimental structure determination, etc.), but proteins with lower scores need not be discarded until further information is obtained regarding the protein's function.

Finally, the potential of the active site profiles for sub-family functional classification of sequences was demonstrated using the protein kinase family as an example. Typically, protein families are classified based on similarities across the entire sequence or global structure. The advantage of the instant classification method is that it focuses on the region of the protein responsible for both enzymatic function and specificity. While the kinases all perform the same enzymatic function, individual family members must recognize their particular biological substrate or substrates. The recent development of specific kinase inhibitors (Traxler, et al. (2001), Med. Res. Rev., vol. 21(6), 499-512; Woolfrey & Weston (2002), Curr. Pharm. Des., vol. 8(17), 1527-45) as cancer treatments demonstrates that such biological specificity can be translated into therapeutic specificity. The regions of conservation and variability found in the active site profiles of functional families can provide important information for selective and specific inhibitor design.

4. Methods

A. FFF Construction and Decoy Identification

The FFFs were in this study were constructed as described above. Each FFF related key residues for the corresponding function by way of encoding spatial relationships based upon alpha carbon positions. The allowable geometric ranges of these distances was determined empirically to be both structurally distinct with respect to similar arrangements observed in structures available in the PDB that posses the same activity, yet sufficiently “fuzzy” to identify all true positive structures in the PDB. In this way, the FFFs were maximally inclusive for their respective functions, yet structurally unique.

Decoys for functional families, defined by a particular FFF, were identified by assessing all PDB structures that possess the key functional residues within a similar range of geometric distances to the true positive structures. Although each FFF had been tailored to be specific and selective in identifying all members of a functional family, instances were explored in which the key functional residues were present in other proteins, but in a three dimensional arrangement that precluded function. Such proteins, containing geometrically similar yet non-functioning sites, are “decoy proteins.” For these studies, representative structures for decoy proteins were chosen because they had the most similar geometric distances between key FFF-recognized residues compared to the ensemble of true positive structures.

B. Construction of Active Site Profiles

Active site profiles were generated for each FFF by collecting the subset of protein sequences known to perform the function described by the FFF and for which there were experimentally determined structures. For each structurally characterized protein belonging to the functional family, all residues within a default 10 Å radius from the key functional residues defined by the FFF were extracted (whether protruding individually into the pre-defined spatial environment or as part of a peptide sequence within the spatial environment). In this case, distances were measured based on alpha carbon positions, although other radii, as well as distance measured between other atoms (including pseudoatoms) can be employed, as are left to the discretion of the skilled artisan. Each extracted amino acid and/or fragment represented a potential substructure motif that maps a portion of each protein's active site.

Construction of the particular active site profile proceeded by annealing the neighboring fragments in a sequence-ordered manner to yield a single string composed of the discontinuous subsets. The strings from each structure were aligned with CLUSTALW Version 1.8 (Thompson, et al. (1994), Nucleic Acids Res., vol. 22(22), 4673-80) using default values except for the following parameters: GAPEXT (gap extension penalty)=1.0 and MATRIX=ID. Using these non-default parameters guided the program toward alignments that created scores weighted towards identities, but still allowed for gaps to accommodate diversity among larger protein families.

FFFs were used to recognize active sites on computationally determined protein structures. For this study, the PROSPECTOR threading algorithm was used to analyze query proteins with no known or available experimental structures. To extract the analogous active site signature from a query protein sequence, the residue positions from the threading template structure were utilized and transferred to the residue identities from the threaded sequence to form its active site signature.

C. Scoring of Active Site Signatures

An active site profile alignment was used to calculate the profile score, which measured the similarity of the active site signatures of the protein family members. Once the functional site profile and score were generated for a functional family, the corresponding active site signature from a query protein was compared to the profile to quantify its relationship to the protein family. In this example, query sequences and their associated structures, either experimentally determined or generated by computational methods, were recognized by an FFF. The query sequence active site signature, as defined by distance to the FFF key functional residues, was extracted and added to the set of family member's active site signatures. These new alignments were generated using CLUSTALW, and a new score (a “family” score) was calculated. Comparison of the family score, with the query signature included, to the profile score indicates how well the query sequence fits within the scope of observed active site variation for a family of functionally related proteins.

In addition to the family score for a query protein sequence, a pairwise score was also calculated. Whereas the family score indicates the similarity between the threaded sequence and the whole functional family of sequences with known structures, the pairwise score was derived from the pairwise alignment of the query active site signatures and each individual protein of the functional family. Following the pairwise alignment and scoring of this query signature to each family member signature, the score for the best alignment among the alignments to all family members was retained. Thus, the pairwise score identified the structurally characterized family member that was most closely related to the query sequence active site.

Scores were generated by considering each functional site residue position in the corresponding active site profile. The variation of residue types for each functional site residue was evaluated for the following four conditions, as assigned by CLUSTALW: identity; strongly conserved; weakly conserved; and gapped positions. Value assignments for these parameters were derived empirically such that identities dominate the score, and the assignments are as follows: identity=+1.0, strongly conserved=+0.2, weakly conserved=+0.1, and gapped=−0.5. The values at each residue position are summed to generate a score that is then normalized by the number of positions in the active site profile, as indicated by equation 1.

$$Score = \frac{{\sum }_{1}^{n} {S}_{I} + {\sum }_{1}^{m} {S}_{S} + {\sum }_{1}^{k} {S}_{W} + Gaps}{N} \qquad ( 1 )$$

S_{I} is the score for positions that are fully conserved, S_{S} is the score for the positions that are strongly conserved, S_{W} is the score for the positions that are weakly conserved, and N is the number of residues in the functional site profile. For a gap-free alignment of functional sites, the score varies from 0 to 1. When gaps are introduced, the score can fall below zero.

D. Genome Analysis

Over 25,500 human protein sequences were downloaded from Build 22 of NCBI's RefSeq database. Pruitt & Maglott (2001), Nucleic Acids Res 29(1), 137-40. These sequences were threaded to a structure library derived from the 092 release of the PDB using the PROSPECTOR algorithm. Long sequences were broken into overlapping fragments of 150 residues, and both the fragments and the full sequence were threaded individually. The FFFs were applied to the resulting structure models, and the results stored in a relational database.

E. Consensus Functional Annotations for Benchmarking of Active Site Profile Scores

To establish sets of protein sequences used in benchmarking the active site profile scores, equivalent Blocks, PRINTS, and Pfam motifs were identified for each FFF. The Blocks, PRINTS, and Pfam tools and motif libraries were applied to the sequences of functional family members of known structure using the default cutoffs for each tool (Blocks: E-value of 5; PRINTS: the top ten hits; Pfam: E-value of 10). The versions of the motif libraries and tools used are as follows: the Blocks+ library was downloaded on Nov. 15, 2000, and searched using BLIMPS version 3.4.0 (Wallace & Henikoff (1992), Comput Appl Biosci., vol. 8(3), 249-54), the PRINTS 30.0 library was searched using FingerPRINTScan version 3.595 (Scordis, et al. (1999), Bioinformatics, vol. 15(10), 799-806), and the Pfam 6.3 library was searched with HMMER 2.1.1 (Eddy (1998), Bioinformatics, vol. 14(9), 755-63).

The list of motifs that match any of the structurally characterized family members was examined, and motifs that covered the same family as that covered by the FFF were identified. Motifs that covered a wholly included subfamily of the functional family described by the FFF were also identified. These equivalencies between FFFs and public tool motifs were stored in a relational database.

The genomic sequences were analyzed using the same versions of the Blocks, PRINTS, and Pfam motif libraries and tools, and the results were stored in a relational database. The stored FFF-public tool motif equivalencies were then used to automatically select a subset of sequences for which the FFF and the public tools all provided the same functional assignment. This subset was designated as the pool of potential consensus annotated sequences.

The results of a Blast analysis of the pool of potential consensus annotated sequences were then analyzed. Blast version 2.1.2 was run, using the GenBank non-redundant sequence set downloaded on May 25, 2001. The Blast results for each potential consensus annotated sequence were analyzed. Only Blast hits with an E-value of at least 10⁻² were considered. Sequences for which Blast identified a known member of the family covered by the FFF and the Blocks, PRINTS, and Pfam motifs were identified as a consensus annotated sequences.

All patents, publications, scientific articles, web sites, and other referenced materials mentioned in this specification are indicative of the levels of skill of those skilled in the art to which the invention pertains, and each such referenced material is hereby incorporated by reference to the same extent as if each reference had been incorporated by reference in its entirety individually. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments of the present invention, any of the terms “comprising”, “consisting essentially of”, and “consisting of” may be replaced with either of the other two terms. Also, the terms “comprising”, “including”, “containing”, etc. are to be read expansively and without limitation. It must be noted that as used herein and in the appended claims, the singular forms “a,” “an,” and “the” include plural reference unless the context clearly dictates otherwise. Thus, for example, a reference to “a host cell” includes a plurality (e.g., a culture or population) of such host cells, and so forth.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Other embodiments are within the following claims.

What is claimed is:

1. A method for creating a functional site profile for a protein functional site that confers a particular biochemical function, comprising:
a. obtaining a protein model that represents a plurality of amino acid residues in a spatial environment of a protein functional site;
b. identifying in the protein model at least two non-contiguous amino acid residues in the plurality of amino acid residues in the spatial environment of the protein functional site; and
c. assembling the non-contiguous amino acids identified in part (b) into a functional site profile.

2. A method according to claim 1 wherein the protein functional site is selected from the group consisting of an active site, a ligand binding site, and a protein-protein interaction site.

3. A method according to claim 1 wherein the particular biochemical function is a catalytic function.

4. A method according to claim 1 wherein the protein model is selected from the group consisting of a high resolution model, a moderate resolution model, and a low resolution model.

5. A method according to claim 1 wherein the protein model is an approximate model.

6. A method according to claim 1 wherein the protein model represents an experimentally determined three-dimensional structure for the complete protein.

7. A method according to claim 1 wherein the protein model represents a three-dimensional structure for a domain of the protein that exhibits a particular biochemical function.

8. A method according to claim 1 wherein the plurality of amino acid residues contains between 2 and about 300 amino acid residues.

9. A method according to claim 1 wherein the spatial environment represents a volume defined by at least one polyhedron.

10. A method according to claim 9 wherein the volume is defined by a plurality of polyhedrons.

11. A method according to claim 9 wherein the volume is defined by a union of a plurality of polyhedrons.

12. A method according to claim 9 wherein the polyhedron is a sphere.

13. A method according to claim 12 wherein the sphere has a radius of less than about 30 Å.

14. A method according to claim 12 wherein the sphere has a radius of about 10 Å.

15. A method according to claim 10 wherein the volume is centered on a representation of an amino acid residue in the protein functional site.

16. A method according to claim 11 wherein each of the polyhedrons comprising the volume is a sphere.

17. A method according to claim 16 wherein each of the spheres has the same radius.

18. A method according to claim 17 wherein each radius is about 10 Å, and each sphere is centered on a representation of a different amino acid residue in the protein functional site.

19. A method according to claim 15 wherein the amino acid residue being represented is an amino acid residue of a functional site descriptor.

20. A method according to claim 1 wherein in part (b) the at least one of the non-contiguous amino acid residues is contained within a peptide fragment comprised of at least two contiguous amino acid residues within the spatial environment of the protein functional site.

21. A method according to claim 1 wherein the amino acid residues of the functional site profile are ordered as the amino acid residues appear in a linear representation of the amino acid sequence of the protein.

22. A method according to claim 1 further comprising creating a consensus functional site profile for a particular biochemical function.

23. A method according to claim 22 wherein the consensus functional site profile is created by:
a. generating an independent functional site profile for each of a plurality of different proteins comprising different amino acid sequences but having the same biochemical function; and
b. developing a consensus functional site profile by comparing the functional site profiles.

24. A method according to claim 23 wherein the comparison of part (b) is performed by aligning a plurality of different amino acid sequences.

25. A method according to claim 1 that is automated.

26. A computer program product comprising a computer useable medium having computer program code logic recorded thereon for performing the method of claim 1.

27. A computer program product comprising a computer useable medium having a functional site profile for a protein functional site stored thereon.

28. A computer program product according to claim 27 that comprises a plurality of functional site profiles.

29. A computer program product according to claim 27 wherein each of the functional site profiles corresponds to a different biochemical function.

30. A computer program product according to claim 27 that comprises a plurality of independent functional site profiles for one biochemical function.

31. A computer program product according to claim 27 wherein the functional site profile is a consensus functional site profile.

32. A method of determining if a protein has a particular biochemical function, comprising:
a. obtaining an amino acid sequence for a protein of interest;
b. analyzing the amino acid sequence of the protein of interest with a functional site profile for a particular biochemical function to determine if the functional site profile exists in the amino acid sequence of the protein of interest, wherein the functional site profile comprises at least two non-contiguous amino acid residues in a plurality of amino acid residues in a spatial environment of a protein functional site that confers the particular biochemical function assembled into a contiguous sequence of amino acid residues; and
c. if so, making a determination that the protein of interest has the particular biochemical function.

33. A method according to claim 32 that is automated.

34. A method according to claim 32 wherein the analysis of part (b) is performed by aligning the functional site profile with the amino acid sequence of the protein of interest.

35. A method according to claim 34 wherein the determination of whether the functional site profile exists in the amino acid sequence of the protein is made using a scoring function that evaluates if the alignment between the portion of the amino acid sequence of the protein and the functional site profile is indicative of the existence of the functional site profile in the protein.

36. A method according to claim 32 that is applied to a plurality of proteins of interest.

37. A method according to claim 32 that is performed using a plurality of functional site profiles.

38. A method according to claim 32 wherein the plurality of functional site profiles represents a plurality of different biochemical functions.

39. A method of classifying a protein based on biochemical function, comprising:
a. obtaining an amino acid sequence for a protein of interest;
b. analyzing the amino acid sequence of the protein of interest with a functional site profile for a particular biochemical function to determine if the functional site profile exists in the amino acid sequence of the protein of interest, wherein the functional site profile comprises at least two non-contiguous amino acid residues in a plurality of amino acid residues in a spatial environment of a protein functional site that confers the particular biochemical function assembled into a contiguous sequence of amino acid residues; and, if so,
c. classifying the protein of interest as having the biochemical function corresponding to the functional site profile.

40. A method of identifying a functional site in a protein, comprising:
a. obtaining an amino acid sequence for a protein of interest;
b. analyzing the amino acid sequence of the protein of interest with a functional site profile for a particular biochemical function to determine if the functional site profile exists in the amino acid sequence of the protein of interest, wherein the functional site profile comprises at least two non-contiguous amino acid residues in a plurality of amino acid residues in a spatial environment of a protein functional site that confers the particular biochemical function assembled into a contiguous sequence of amino acid residues; and, if so,
c. identifying the amino acid residues in the protein of interest that correspond to the functional site profile, thereby identifying the functional site.

41. A method according to claim 40 that is automated.

42. A method according to claim 40 wherein the functional site profile is a consensus functional site profile.

43. A method according to claim 40 that is applied to a plurality of proteins of interest.

44. A method of identifying a compound that specifically interacts with a protein, comprising:
a. identifying a functional site in a target protein in accordance with the method of claim 40; and
b. using information for the amino acid residues of the functional site to identify a compound that specifically interacts with the functional site of the target protein.

45. A method according to claim 44 that is automated.

46. A method of claim 44 that further comprises identifying the structure of the functional site identified using the functional site profile.

47. A method according to 46 that further comprises using the structure of the functional site to identify a compound that specifically interacts with the functional site of the target protein.

48. A method according to claim 44 wherein, in addition to identifying the functional site in the target protein, functional sites corresponding to the functional site profile are also identified in other members of the target protein's protein family.

49. A method according to claim 48 wherein information for the functional sites corresponding to the functional site profile in other members of the target protein's protein family is used to identify compounds that interact with the target protein and at least one other member of the target protein's protein family.

50. A method according to claim 48 wherein information for the functional sites corresponding to the functional site profile in other members of the target protein's protein family is used to identify compounds that interact with the target protein but not other members of the target protein's protein family.

51. A compound identified according to the method of claim 44 that specifically interacts with the target protein.

52. A method of creating an inverse pharmacophore, comprising:
a. identifying amino acid residues that comprise a functional site in a target protein in accordance with the method of claim 40;
b. obtaining a three-dimensional structural representation of the functional site of part (a);
c. using information from parts (a) and (b) to create an inverse pharmacophore for the functional site of the target protein.

53. A method of identifying a compound, comprising:
a. obtaining an inverse pharmacophore made in accordance with claim 52;
b. generating a pharmacophore that is complementary to the inverse pharmacophore; and
c. screening a library of chemical structures to identify a compound that matches the pharmacophore.

54. A compound identified according to the method of claim 53.
